(19) United States
(12) Patent Application Publication
(10) Pub. No.: US 2022/0023269 A1
(43) Pub. Date: Jan. 27, 2022

(54) Methods Of Treating Diabetes In Severe Insulin-Resistant Diabetic Subjects

(71) Applicant: Balticgruppen Bio AB, Umeä (SE)

(72) Inventors: Helena EDLUND, Umeä (SE); Björn ERIKSSON, Umeä (SE)

(21) Appl. No.: 17/291,358

(22) PCT Filed: Nov. 5, 2018
(86) PCT No.: PCT/GB2018/053203

§ 371 Date: May 5, 2021

(51) Int. Cl. A61K 31/433; A61K 31/7042; A61P 3/10

(52) U.S. Cl. CPC A61K 31/433; A61P 3/10; A61K 31/7042

(57) ABSTRACT

This invention relates to a new method of treating diabetes in a population of subjects that is characterised as having severe insulin-resistant diabetes. This population is typically obese, insulin resistance and hyperglycemic and has an elevated risk of diabetic kidney disease. The compound of formula I has been found to treat high body weight, insulin resistance and hyperglycemia and to have a positive effect on microvascular perfusion in glomeruli and so is particularly suited for the treatment of this patient group.

### FIELD OF THE INVENTION

The present invention relates to the use of an AMPK activator in the treatment of diabetes in patients that are particularly suited to this treatment. Suitable patients are characterised by having increased insulin resistance and a high body weight. In particular, the treatment is useful for treating type 2 diabetes in patients with severe insulin-resistant diabetes.

### BACKGROUND OF THE INVENTION

Diabetes comprises two distinct diseases, type 1 (or insulin-dependent diabetes) and type 2 (insulin-independent diabetes), both of which involve the malfunction of glucose homeostasis. Type 2 diabetes currently affects more than 400 million people in the world and this number is rising rapidly. Complications of type 2 diabetes include severe cardiovascular problems, kidney failure, peripheral neuropathy, blindness and even loss of limbs and, ultimately, death in the later stages of the disease. Type 2 diabetes is characterised by insulin resistance, and there is presently no definitive cure. Most treatments used today are focused on remedying dysfunctional insulin signalling, inhibiting glucose output from the liver or inhibiting reabsorption of glucose in the kidney but many of those treatments have several drawbacks and side effects. Although there have been improvements in long-term outcomes, the excess mortality and cardiovascular morbidity remain a considerable challenge for healthcare systems.

The current front-line therapy for type 2 diabetes is metformin, a biguanide that lowers plasma glucose primarily by reducing hepatic glucose production. Nevertheless, there remains a need for treatments for subjects with type 2 diabetes who do not achieve glycaemic control with metformin.

Diabetes is presently classified into two main forms, type 1 and type 2 diabetes, but type 2 diabetes in particular is highly heterogeneous.

Existing treatment guidelines are limited by the fact they respond to poor metabolic control when it has developed, but do not have means to predict which patients will need intensified treatment. Evidence suggests that early treatment is crucial for prevention of life-shortening complications because target tissues seem to remember poor metabolic control decades later (Emma Ahlqvist et. al., The Lancet Diabetes Endocrinology, Vol. 6, No. 5, p361-369, 2018).

There remains a need to identify a better classification for such patients to provide a mechanism to identify individuals with increased risk of complications at diagnosis and enable individualised treatment regimens.

We have now found a new treatment that is surprisingly effective for patients suffering from severe insulin-resistant diabetes.

The listing or discussion of an apparently prior-published document in this specification should not necessarily be taken as an acknowledgement that the document is part of the state of the art or is common general knowledge.

### DISCLOSURE OF THE INVENTION

According to a first aspect of the invention, there is provided a method of treating diabetes, said method comprising administering a compound of formula I,

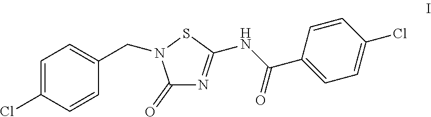 [SMILES: O=C(Nc1nc(=O)n(Cc2ccc(Cl)cc2)s1)c1ccc(Cl)cc1]

or a pharmaceutically acceptable salt, solvate or prodrug thereof, to a subject in need thereof, wherein the subject is identified as having severe insulin-resistant diabetes.

The method of the first aspect of the invention is hereinafter referred to as a “method of the invention”.

Subjects that are identified as having severe insulin-resistant diabetes represent a subgroup of subjects suffering from diabetes who are obese, insulin resistant and hyperinsulinaemic. These subjects have a fivefold higher risk of developing diabetic kidney disease compared to other diabetic subjects. There is currently a lack of efficient treatment, and the methods of the invention are particularly suited to these subjects.

Pharmaceutically acceptable salts that may be mentioned include acid addition salts and base addition salts. Such salts may be formed by conventional means, for example by reaction of a free acid or a free base form of the compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of the compound of formula I in the form of a salt with another counter-ion, for example using a suitable ion exchange resin.

Examples of pharmaceutically acceptable addition salts include those derived from mineral acids, such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

The term “prodrug” of a relevant compound of formula I includes any compound that, following oral or parenteral administration, is metabolised in vivo to form that compound in an experimentally-detectable amount, and within a predetermined time (e.g. within a dosing interval of between 6 and 24 hours (i.e. once to four times daily)). Prodrugs of the compound of formula I include derivatives that have, or provide for, the same biological function and/or activity as any relevant compound. For the avoidance of doubt, the term “parenteral” administration includes all forms of administration other than oral administration.

Prodrugs of the compound of formula I may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula I wherein an amino or carbonyl group in the compound of formula I is bonded to any group that may be cleaved in vivo to regenerate the free amino or carbonyl group, respectively.

Examples of prodrugs include, but are not limited to, esters groups of carboxyl functional groups, N-acyl derivatives and N-Mannich bases. General information on prodrugs may be found e.g. in Bundegaard, H. “Design of Prodrugs” p. I-92, Elsevier, New York-Oxford (1985).

The compound of formula I, as well as pharmaceutically-acceptable salts, solvates and prodrugs of said compound are, for the sake of brevity, hereinafter referred to together as the “the compound of formula I”.

The compound of formula I may exist as regioisomers and may also exhibit tautomerism. All tautomeric forms and mixtures thereof are included within the scope of the invention.

All individual features (e.g. preferred features) mentioned herein may be taken in isolation or in combination with any other features (including preferred features) mentioned herein (hence, preferred features may be taken in conjunction with other preferred features, or independently of them).

The compound of formula I is a direct PAN-AMPK activator that does not enter the brain. In preclinical models of hyperglycaemia/diabetes, the compound of formula I has been found to increase glucose uptake in skeletal muscle, reduce insulin resistance and promote β-cell rest. The compound of formula I increases energy expenditure and prevents/reduces obesity. Like exercise, the compound of formula I lowers blood pressure and increases microvascular perfusion, activates AMPK in the heart, increases cardiac glucose uptake, reduces cardiac glycogen levels, and improves left ventricular stroke volume and endurance. Further, the compound of formula I does not cause cardiac hypertrophy in mouse or in rat. Crucially, therefore, the compound of formula I exhibits a combination of beneficial metabolic and cardiovascular effects that are not observed with any other available anti-diabetic drug.

According to an alternative first aspect of the invention, there is provided the compound of formula I (as defined above), or a pharmaceutically acceptable salt, solvate or prodrug thereof, for use in treating diabetes in a subject identified as having severe insulin-resistant diabetes.

According to a further alternative first aspect of the invention, there is provided the use of the compound of formula I (as defined above), or a pharmaceutically acceptable salt, solvate or prodrug thereof, in the manufacture of a medicament for treating diabetes in a subject identified as having severe insulin-resistant diabetes.

Diabetes is often associated with a variety of symptoms, including polyphagia, polydipsia, polyuria, kidney damage, neurological damage, cardiovascular damage, damage to the retina, damage to the lower limbs, fatigue, restlessness, weight loss, poor wound healing, dry or itchy skin, erectile dysfunction, cardiac arrhythmia, coma and seizures.

By the terms “treat,” “treating,” or “treatment of” (and grammatical variations thereof) it is meant that the severity of the subject's condition is reduced, at least partially improved and/or that some alleviation, mitigation or decrease in at least one clinical symptom is achieved and/or there is a delay in the progression of the disease or disorder. In this respect, these terms may refer to at least a partial reduction in the severity of at least one of the subject's clinical symptoms and/or a reduction in the duration of at least one of said symptoms. The terms “treat,” “treating,” and “treatment of” may also refer to achieving a reduction of blood glucose levels (for example, to or below about 10.0 mmol/mL (e.g. to levels in the range of from about 4.0 mmol/L to about 10.0 mmol/L), such as to or below about 7.5 mmol/mL (e.g. to levels in the range of from about 4.0 mmol/L to about 7.5 mmol/L) or to or below about 6 mmol/mL (e.g. to levels in the range of from about 4.0 mmol/L to about 6.0 mmol/L)). In particular embodiments, in the case of type 2 diabetes, the term may refer to achieving a reduction of blood glucose levels.

A “subject in need” of the methods of the invention includes a subject that is suffering from diabetes, particularly type 2 diabetes. Thus, in one embodiment, the method of the invention is a method of treating type 2 diabetes.

A “therapeutically effective amount”, an “effective amount” or a “dosage” as used herein refers to an amount of a compound, composition and/or formulation that is sufficient to produce a desired effect, which can be a therapeutic and/or beneficial effect. The effective amount or dosage will vary with the age or general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, a “therapeutically effective amount”, “effective amount” or “dosage” in any individual case can be determined by one of skill in the art by reference to the pertinent texts and literature and/or by using routine experimentation. Those skilled in the art will appreciate that the therapeutic effects need not be complete or curative, as long as some benefit is provided to the subject.

As used herein, the terms “disease” and “disorder” (and, similarly, the terms condition, illness, medical problem, and the like) may be used interchangeably.

The distinct population of diabetic sufferers defined as those with “severe insulin-resistant diabetes” (SI RD) lack efficient treatment options. The inventors have found that the clinical and pharmacokinetic effects that are observed for the compound of formula I are particularly suited to the therapeutic needs of diabetic sufferers with severe insulin-resistant diabetes. By directing treatments using the compound of formula I to these patients, significant clinical benefits can be realised, including reduced organ morbidity and increased survival rates.

The term “insulin-resistant” refers to a subject having normal, or in some cases increased, insulin production but significantly reduced insulin sensitivity. Subjects may be categorised as having severe insulin-resistant diabetes according to the criteria set out in Emma Ahlqvist et. al., The Lancet Diabetes Endocrinology, Vol. 6, No. 5, p361-369, 2018. In the study described therein, subjects were grouped based on six variables (glutamate decarboxylase antibodies, age at diagnosis, BMI (body mass index), HbA_{1c}, and homoeostatic model assessment 2 estimates of β-cell function and insulin resistance), and were related to prospective data from patient records on development of complications and prescription of medication. Subjects having severe insulin-resistant diabetes typically have a high BMI, such as at least 30 kg/m² or particularly at least 35 kg/m². Subjects may also have a HbA_{1c} level of at least 52 mmol/mol. Still further, subjects may have C-peptide levels that are above the reference range at the particular test site. Determination of each of these clinical parameters can be easily achieved using routine methods that are known to the skilled person.

It has been found that treatment with the compound of formula I can reduce bodyweight, ameliorate insulin resistance, and treat hyperglycemia in mice. Administration of the compound of the invention to diet-induced obese mice increased glucose uptake in skeletal muscle, reduced β cell stress, and promoted β cell rest.

Administration of this compound to humans has also been found to have a positive effect on microvascular perfusion in glomeruli. The compound reduced fasting plasma glucose levels and homeostasis model assessment of insulin resistance (HOMA-IR) in a phase IIa clinical trial in type 2 diabetes (T2D) patients on Metformin. The compound also improved peripheral microvascular perfusion and reduced blood pressure both in animals and type-2 diabetic patients. Administration of the compound of formula I, or a pharmaceutically acceptable salt, solvate or prodrug thereof, is therefore expected to result in beneficial effects in subjects suffering from diabetes, particular severe insulin-resistant diabetes.

These effects may include a reduction in the body weight of the subject. For example, the body weight of the subject may be reduced such that the subject is no longer considered obese. A patient may be determined to be obese if they have a BMI of 30 kg/m² or higher. This includes “Obese Class I” or “moderately obese” where the BMI is from 30 to 35 kg/m², under the WHO categorisation system. At higher BMI levels, a patient may be classified as being severely obese (Obese Class II; BMI of from 35 to 40 kg/m²), very severely obese (Obese Class III; BMI of from 40 to 45 kg/m²), morbidly obese (Obese Class IV; BMI of from 45 to 50 kg/m²), or having a still higher degree of obesity. Treatment of such patients using the methods of the invention may therefore result in the patient being classified in a lower weight category, and may even result in the BMI of the patient being reduced below the threshold for obesity. Patients having a BMI of from 25 to 30 kg/m² are typically classified as overweight and may benefit from the present therapy, although they may not be classified as having severe insulin-resistant diabetes. In a particular embodiment of the invention, the bodyweight of the subject is reduced.

The methods of the invention are therefore particularly suited to the treatment of subjects that are obese. In the context of the present invention, unless otherwise specified, the term “obese” includes subjects that are classified in Obese Class I and above according to the WHO classification system. Thus, in embodiment the method is performed on a subject that has a BMI of at least 30 kg/m². In another embodiment of the invention, the body mass index of the subject is reduced, e.g. so that the patient is categorised as being in an obesity class of lower severity or categorised as not being obese at all.

As used herein, references to a subject (or to subjects) refer to a living subject being treated, or receiving preventative medicine, including mammalian (e.g. human) subjects. In particular, references to a subject refer to a human subject.

The methods of the invention may give rise to other beneficial effects for the subject being treated. For example, the compound of the invention has been shown to have positive effects on renal hemodynamics in patients suffering from type-2 diabetes. The compound may cause a rapid, stable and reversible reduction in estimated glomerular filtration rate (eGFR) in patients that is consistent with reduced intraglomerular pressure. This is indicative of an early hemodynamic effect. The method of the invention may therefore improve the renal hemodynamics for the subject. More specifically, the method of the invention may result in a reduction, e.g. a clinically therapeutic reduction, in the intraglomerular pressure as may be determined via a reduction in estimated glomerular filtration rate (eGFR) in the subject.

Other clinical benefits include a reduction in organ morbidity and an increase in the likelihood of survival over a given time period following diagnosis.

Subjects may be categorised as having severe insulin-resistant diabetes as described above. Particular subjects for which treatment by the method of the invention may be especially effective include those having a HbA_{1c} level of at least 52 mmol/mol. This value may be determined using routine methods known in the art.

Other particular subjects that may be mentioned include those having C-peptide levels that are above the reference range at the particular test site. C-peptide is a short 31-amino-acid polypeptide that connects insulin's A-chain to its B-chain in the proinsulin molecule. In diabetes, a measurement of C-peptide blood serum levels can be used to distinguish between certain diseases with similar clinical features. The reference range may vary depending on the patient and their recent activity, such as any recent food intake. For example, a C-peptide measurement in a healthy individual after fasting may be in the range of 0.13 to 0.70 nmol/L. Particular elevated values that may be mentioned in the context of a subject having severe insulin-resistant diabetes include a blood C-peptide concentration of at least 1.4 nmol/L, more particularly at least 1.5 nmol/L.

Subjects that are characterised as having severe insulin-resistant diabetes may have an increased risk of susceptibility to diabetic kidney disease, or they may be diagnosed with diabetic kidney disease. They may also have, or have an increased risk for, cardiovascular disease. The methods of the present invention are believed to provide at least some organ protective benefits to patients, particularly those indicated here. As a consequence, treatment of subjects characterised as having severe insulin-resistant diabetes according to the methods of the present invention may result in a lessening of the prevalence of diabetic kidney disease during and/or following treatment. Thus, it is preferred that the subject to be treated has an increased risk of susceptibility to diabetic kidney disease. Subjects that already have diabetic kidney disease may also benefit from the present methods of treatment, for example by way of a reduction in the rate at which the severity of the diabetic kidney disease increases. In an embodiment of the invention, the subject to be treated has diabetic kidney disease.

The treatment of a subject having severe insulin-resistant diabetes requires that the subject is first identified as having that condition. Some of the clinical parameters necessary for diagnosis are described elsewhere herein and also in Emma Ahlqvist et. al., The Lancet Diabetes Endocrinology, Vol. 6, No. 5, p361-369, 2018. The present invention therefore also relates to a method of treating diabetes, said method comprising
(i) identifying a subject having severe insulin-resistant diabetes; and
(ii) administering a compound of formula I (as defined elsewhere herein), or a pharmaceutically acceptable salt, solvate or prodrug thereof, to said subject.

Step (i) involves the clinical evaluation of the subject, including an assessment of at least one of the physiological and pathological aspects described above for these subjects. A subject is considered to have severe insulin-resistant diabetes if they satisfy the criteria set out in Ahlqvist et. al., ibid. This may include, for example, one or preferably all of the following: a BMI of at least 30 kg/m², a HbA_{1c} level of at least 52 mmol/mol and C-peptide levels that are above the reference range at the particular test site.

Step (ii) may be carried out using any appropriate administration route, formulation and dosage regime, including those described elsewhere herein. Said treatment may result in, for example, the suppression of the development of systemic insulin resistance in the subject. Said treatment may also result in the inducement of body weight loss and body fat loss, potentially without reducing food intake by the subject. Other clinical effects that may arise from the treatment will be apparent from the examples.

The compound of formula I is also known as 4-chloro-N-[2-[(4-chlorophenyl)methyl]-3-oxo-1,2,4-thiadiazol-5-yl]benzamide.

Where it is possible for a compound to exist as a tautomer the depicted structure represents one of the possible tautomeric forms, wherein the actual tautomeric form(s) observed may vary depending on environmental factors such as solvent, temperature or pH.

The compound of formula I, and pharmaceutically acceptable salts, solvates and prodrugs thereof, may be prepared in accordance with techniques that are well known to those skilled in the art, for example as described hereinafter. For example, 4-chloro-N-[2-[(4-chlorophenyl)methyl]-3-oxo-1,2,4-thiadiazol-5-yl]benzamide may be made in accordance with the techniques described in international patent application WO 2011/004162, and all of its content is hereby incorporated by reference.

The compound of the invention may therefore be administered to a subject in any form which facilitates a reduction in both fasting plasma glucose levels and insulin resistance (e.g. according to the homeostasis model assessment of insulin resistance). In particular, the compound of the invention may be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. In particular embodiments, the compound of formula I, or pharmaceutically acceptable salt, solvate or prodrug thereof, is administered orally, nasally, parenterally or by inhalation. Preferably, the administration occurs orally.

The compound of the invention will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science, 249, 1527 (1990).

In the methods of the invention described herein, the pharmaceutically active compounds may be administered by way of known pharmaceutical formulations, including tablets, capsules or elixirs for oral administration, suppositories for rectal administration, sterile solutions or suspensions for parenteral or intramuscular administration, or via inhalation, and the like. Administration through inhalation is preferably done by using a nebulizer, thus delivering the compound of the invention to the small lung tissue including the alveoli and bronchioles, preferably without causing irritation or cough in the treated subject.

The preparation of other suitable formulations may be achieved non-inventively by the skilled person using routine techniques and/or in accordance with standard and/or accepted pharmaceutical practice.

The amount of compound of the invention that is administered to the subject will depend on the condition to be treated or prevented, the severity of the condition, the subject, and the route of administration, as well as the compound(s) which is/are employed, but may be determined non-inventively by the skilled person. The compound of the invention may be administered at varying therapeutically effective doses to a patient in need thereof.

Although doses will vary from patient to patient, suitable daily doses are in the range of about 0.1 to about 5000 mg (e.g., 0.1, 0.5, 1, 2, 5, 10, 15, 20, 25, 30, 35, 40, 45, 50, 75, 100, 150, 200, 250, 300, 350, 400, 450, 500, 550, 600, 650, 700, 750, 800, 850, 900, 950, 1000 mg, 1250 mg, 1500 mg, 1750 mg, 2000 mg, 2500 mg, 3000 mg, 3500 mg, 4000 mg, 4500 mg, 5000 mg, and the like, or any range or value therein) per patient, administered in single or multiple doses. Administration may be continuous or intermittent (e.g. by bolus injection). The dosage may also be determined by the timing and frequency of administration. In the case of oral or parenteral administration the dosage will preferably vary from about 1 mg to about 2000 mg per day of a compound of the invention (or, if employed, a corresponding amount of a pharmaceutically acceptable salt or prodrug thereof). In particular embodiments, the compound of formula I, or pharmaceutically acceptable salt, solvate or prodrug thereof, is administered to a subject at a daily dose in the range of from about 1 to about 2000 mg.

The term “about,” as used herein when referring to a measurable value such as an amount of a compound, dose, time, temperature, and the like, refers to variations of 20%, 10%, 5%, 1%, 0.5%, or even 0.1% of the specified amount.

In any event, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

The medical practitioner, or other skilled person, will be able to determine routinely the actual dosage which will be most suitable for an individual patient. The above-mentioned dosages are exemplary of the average case; there can, of course, be individual instances where higher or lower dosage ranges are merited, and such are within the scope of this invention.

In addition, in some embodiments, the compound of the invention will be used in combination with one or more other therapeutic medications, or their pharmaceutically acceptable salts, solvates or prodrugs, for manufacturing a medicament for the uses described above (e.g. for treating type 2 diabetes).

Particular other therapeutic medications that may be mentioned in this respect include sodium-glucose transport protein 2 (SGLT2) inhibitors. The skilled person will understand that a sodium-glucose transport protein 2 inhibitor is a substance or agent that inhibits the activity of sodium-glucose transport protein 2. Thus, according to a second aspect of the invention, there is provided a combination of:
(A) a compound of formula I (or a pharmaceutically acceptable salt, solvate or prodrug thereof); and
(B) a sodium-glucose transport protein 2 (SGLT2) inhibitor (or a pharmaceutically acceptable salt, solvate or prodrug thereof).

Said combinations are referred to herein as “combinations of the invention”.

By the phrase “inhibits the activity of sodium-glucose transport protein 2” we mean that the substance or agent elicits a decrease in one or more functions of sodium-glucose transport protein 2, and by decrease in the functions of sodium-glucose transport protein 2 we include the cessation of one or more functions of sodium-glucose transport protein 2, or a reduction in the rate of a particular function. A particular function that may be fully or partially inhibited is the ability of sodium-glucose transport protein 2 to act as a glucose transporter.

SGLT2 inhibitors are substances or agents that selectively inhibit the activity of SGLT2. By selectively inhibits the activity of SGLT2 we mean that the SGLT2 inhibitor selectively ceases or reduces the rate of one or more functions of SGLT2 in preference to one or more functions of sodium-glucose transport protein 1 (SGLT1). For example, the level of selectivity towards SGLT2 over SGLT1 may range from about 2:1 to 5000:1. For example, a SGLT2 inhibitor may have a selectively of about 10:1, about 50:1, about 100:1, about 250:1, about 500:1, about 1000:1, about 5000:1, greater than about 5000:1 for SGLT2 over SGLT1. Thus, in particular embodiments, the SGLT2 inhibitor is a selective SGLT2 inhibitor. The skilled person will be aware of standard tests that can be performed that will allow the skilled person to determine whether a substance or agent acts as a sodium-glucose transport protein 2 inhibitor.

In a preferred embodiment, the sodium-glucose transport protein 2 inhibitor is a so-called “small molecule” with a molecular weight of less than 900 Daltons (Da). Such molecules may be referred to as “drug-like” molecules. In particular embodiments, the sodium-glucose transport protein 2 inhibitor present in the combination of the invention is a gliflozin. Gliflozins are a known class of small-molecule sodium-glucose transport protein 2 inhibitors. Hawley et al. (Diabetes, 2016, 65, 2784-2794) and Villani et al. (Molecular Metabolism, 2016, 5, 1048-1056) have recently discussed the possible mechanisms of action of certain gliflozins. By inhibiting sodium-glucose transport protein 2, gliflozins reduce the extent of the reabsorption of glucose in the kidney (i.e. renal glucose reabsorption) from the glomerular filtrate, which in turn reduces the blood glucose concentration. Any compound that is capable of inhibiting sodium-glucose transport protein 2 may be effective in the combinations of the invention.

Particular sodium-glucose transport protein 2 inhibitors (of the gliflozin class) which may be present in the combination of the invention include, but are not limited to, canagliflozin, dapagliflozin, empagliflozin, ipragliflozin, tofogliflozin, sergliflozin (such as sergliflozin etabonate), remogliflozin (such as remogliflozin etabonate), ertugliflozin and sotagliflozin, and pharmaceutically acceptable salts, solvates and prodrugs thereof. In preferred embodiments of the invention, the sodium-glucose transport protein 2 inhibitor present in the combination of the invention is canagliflozin [also known as (1S)-1,5-anhydro-1-C-(3-{[5-(4-fluorophenyl)thiophen-2-yl]methyl]}-4-methylphenyl)-D-glucitol], or a pharmaceutically acceptable salt, solvate or prodrug thereof.

Combinations of the invention may be particularly useful in treating diabetes in a subject characterised as having severe insulin-resistant diabetes. Thus, in a third aspect of the invention, there is provided a method of treating diabetes in a subject characterised as having severe insulin-resistant diabetes which method comprises the administration of a combination of the invention, as defined herein, to a subject in need thereof.

Similarly, there is provided the use of a combination of the invention, as defined herein, in the manufacture of a medicament for treating diabetes in a subject characterised as having severe insulin-resistant diabetes.

Components (A) and (B) of the combination of the invention (i.e. the compound of formula I and the SGLT2 inhibitor) may be presented either in separate formulations or as a combined preparation (i.e. presented as a single formulation including a compound of formula I and a SGLT2 inhibitor). The compound of formula I and the SGLT2 inhibitor may be administered (optionally repeatedly), either simultaneously, or sufficiently closely in time, to enable a beneficial effect for the subject. Preferably said beneficial effect is greater, over part or all the course of the treatment, than that achievable through the use of a formulation comprising compound of formula I or a formulation comprising the SGLT2 inhibitor, or is a beneficial effect that is not observed when the treatment involves the use of one but not both of the two principal components. Determination of the beneficial effects of the combination of the invention over the course of treatment will depend upon the condition to be treated or prevented, but may be achieved routinely by the skilled person.

Thus, the person skilled in the art will recognise that components (A) and (B) of the combination of the invention may be administered sequentially, separately and/or simultaneously, over the course of treatment of the relevant condition. Administration in this way may be necessary where the two active substances have different pharmacokinetic profiles. For example, the frequency of dosing of one component of the combination may need to be altered separately from the dosing frequency of the other. Therefore, in particular embodiments of the invention, the compound of formula I and the sodium-glucose transport protein 2 (SGLT2) inhibitor are administered sequentially, separately and/or simultaneously to a subject in need thereof.

The compound of formula I may be administered to a subject that has also been (or will be) treated with a sodium-glucose transport protein 2 (SGLT2) inhibitor for the purpose of treating diabetes. Therefore, in another aspect of the invention there is provided the use of a compound of formula I, or a pharmaceutically acceptable salt, solvate or prodrug thereof, in the manufacture of a medicament for the treatment of diabetes in a subject identified as having severe insulin-resistant diabetes, wherein the medicament is administered to a subject that is also treated with a sodium-glucose transport protein 2 (SGLT2) inhibitor (e.g. as defined elsewhere herein), or a pharmaceutically acceptable salt, solvate or prodrug thereof.

Similarly, a sodium-glucose transport protein 2 (SGLT2) inhibitor may be administered to a subject that has also been (or will be) treated with a compound of formula I for the purpose of treating diabetes. Therefore, in another aspect of the invention there is provided the use of a sodium-glucose transport protein 2 (SGLT2) inhibitor (e.g. as defined elsewhere herein), or a pharmaceutically acceptable salt, solvate or prodrug thereof, in the manufacture of a medicament for the treatment of diabetes in a subject identified as having severe insulin-resistant diabetes, wherein the medicament is administered to a subject that is also treated with a compound of formula I, or a pharmaceutically acceptable salt, solvate or prodrug thereof.

Sodium-glucose transport protein 2 inhibitors, such as gliflozins, and pharmaceutically acceptable salts, solvates and prodrugs thereof, may be prepared in accordance with techniques that are well known to those skilled in the art, for example as described hereinafter. For example, canagliflozin, may be made in accordance with the techniques described in international patent application no. WO 2005/012326. For the avoidance of doubt, references to canagliflozin herein include canagliflozin hemihydrate, marketed under the trade name Invokana®. Canagliflozin and other SGLT2 inhibitors may administered at levels according to generally accepted dosages known in the art.

The quantity of the compound of formula I present in the combinations of the invention may be the same as or different from the amount of the SGLT2 inhibitor present. Thus, the weight ratio of the SGLT2 inhibitor to the compound of formula I present in the combination of the invention may be from about 1:1000 to about 1000:1, such as from about 1:100 to 10:1 (e.g. from about 1:10 to about 1:1). Particular weight ratios of the SGLT2 inhibitor to the compound of formula I that may be mentioned include 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10.

The compound of formula I has been shown to treat high body weight, insulin resistance and hyperglycemia in mice and it has been shown to have a positive effect on microvascular perfusion in glomeruli in humans. A distinct and undertreated population of human patients characterised as having severe insulin-resistant diabetes has been identified. This population is typically obese, insulin resistance and hyperglycemic and has an elevated risk of diabetic kidney disease. The benefits of using the compound of formula I in the treatment of diabetes in this population include that the targeting of the therapy in this way will allow for this currently undertreated patient group to receive significantly improved therapeutic attention.

The methods of the invention disclosed herein may also have the advantage that the methods involving the compound of formula I may be more efficacious than, be less toxic than, be longer acting than, be more potent than, produce fewer side effects than, and/or have other useful pharmacological, physical, or chemical properties compared to methods known in the prior art as being useful in the treatment of diabetes in such subjects. Such effects may be evaluated clinically, objectively and/or subjectively by a health care professional, a treatment subject or an observer.

The data summarised herein for the compound of the invention show that the test compound of formula I effectively treats high body weight, insulin resistance and hyperglycemia and has a positive effect on microvascular perfusion in glomeruli.

All patents, patent applications and publications referred to herein are incorporated by reference in their entirety. In the event of conflicting terminology, the present specification is controlling. Further, the embodiments described in one aspect of the present invention are not limited to the aspect described. The embodiments may also be applied to a different aspect of the invention as long as the embodiments do not prevent these aspects of the invention from operating for their intended purpose.

### FIGURES

The following drawings are provided to illustrate various aspects of the present inventive concept and are not intended to limit the scope of the present invention unless specified herein.

FIG. 1(A-F). Test material (“0304”) increases p-T172 AMPK in vitro and increases p-T172 AMPK and ATP in cells. (A and B) Representative immunoblot analysis and quantification of test material dose-dependent suppression of PP2C-mediated dephosphorylation of p-T172 AMPK in absence (A) (n=8 per condition) and presence (B) (n=4 per condition) of 1.0 mM ATP. (C-E) Representative immunoblot analysis (C) and quantification of test material dose-dependent increase of p-T172 AMPK (D) and p-S79 ACC (E) phosphorylation (n=11 per condition) in Wi-38 human lung fibroblast cells. (F) Dose-dependent increase in ATP/protein levels in test material-treated Wi-38 human lung fibroblast cells (n=6 per condition). Data are presented as mean±SEM, *P<0.05, **P<0.01, ***P<(Student's t test).

FIG. 2 (A-I). Test material (“0304”) prevents dysglycemia and insulin resistance in diet-induced obese mice. (A) Timeline in weeks for B6 mice fed a high-fat diet (HFD) and oral gavaged with vehicle or test material±Metformin. (B and C) Fasted glucose (B) and fasted insulin (C) levels in B6 mice on HFD treated with vehicle (n=10), test material (n=10), Metformin (n=10), and test material+Metformin (n=10) for 6 w. (D) HOMA-IR calculations from B and C. (E) Representative immunoblot analysis and quantification of p-T172 AMPK levels in calf muscle of B6 mice on HFD treated with vehicle (n=10), test material (n=10), Metformin (n=10), and test material+Metformin (n=10) for 8 w. (F) Relative mRNA levels of Txnip and Glut1 in calf muscle of B6 mice on HFD treated with vehicle (n=10), test material (n=10), Metformin (n=9), and test material+Metformin (n=10) for 8 w. (G and H) Fasted glucose (G) and insulin (H) levels in B6 mice fed either a regular diet (RD) (n=40) or a HFD for 7 w (=Start; n=10+10). The HFD-fed mice were then continued on HFD and oral gavaged with vehicle (n=10) or test material+Metformin (n=10) for an additional 4 w. (I) HOMA-IR calculations from G and H. Data are presented as mean±SEM, *P<0.05, **P<0.01, ***P<0.001 (Student's t test).

FIG. 3 (A-H). Test material (“0304”) prevents diabetes in hIAPPtg diet-induced obese mice. (A) Timeline in weeks for hIAPPtg mice fed a high-fat diet (HFD) and oral gavaged with vehicle or test material. (B and C) Fasted blood glucose (B) and insulin (C) levels in hIAPPtg mice on HFD treated with vehicle (n=25) and test material (n=27) for 6 w. (D and E) Blood glucose, plasma insulin profiles, and AUC, during i.p. glucose tolerance test (IPGTT) (D) and oral glucose tolerance test (OGTT) (E) in hIAPPtg mice on HFD treated with vehicle (IPGTT, n=13; OGTT, n=7) and test material (IPGTT, n=16; OGTT, n=7) for 6 w. (F) Plasma insulin profiles and AUC during IPGTT of 16 w-old, hIAPPtg mice on HFD treated with test material for 6 w (from D, n=16) compared with that of 10 w-old hIAPPtg mice on regular diet (RD) (n=7). (G) HOMA-IR calculations from glucose and insulin levels from B and C. (H) Matsuda index calculations from IPGTT (D) and OGTT (E) in vehicle and test material-treated hIAPPtg mice. Data are presented as mean±SEM, *P<0.05, **P<0.01 (Student's t test).

FIG. 4 (A-M). Test material (“0304”) dose-dependently averts dysglycemia in diet-induced obese mice and reverts diabetes in hIAPPtg diet-induced obese mice. (A) Representative immunoblot analysis and quantification of p-T172 AMPK levels in calf muscle of CBA mice on high-fat diet (HFD) (n=10) and test material-HFD with 0.4 (n=5), 0.8 (n=10), and 2 mg/g (n=10) test material for 7 w. (B-D) Fasted blood glucose (B) and fasted insulin (C) levels, as well as HOMA-IR (D; from B and C), in CBA mice on HFD (n=10) and test material-HFD with 0.4 (n=5), 0.8 (n=10), and 2 mg/g (n=10) test material for 6 w. (E) Timeline in weeks for hIAPPtg mice fed HFD for 9 w and then either continued on HFD or switched to test material-HFD (2 mg/g in F-H; 0.8 mg/g in I-M) for an additional 7 w. (F-H) Fasted blood glucose (F) and insulin (G) levels, as well as HOMA-IR (H; from F and G), in hIAPPtg mice at start, at 9 w, and 15 w on HFD (n=10), and in hIAPPtg mice at start, at 9 w on HFD, and at 9 w HFD+6 w test material-HFD (2 mg/g) (n=12). (I and J) Body weight (I) and body fat (J) change in hIAPPtg mice on HFD for 15 w (n=12) or HFD for 9 w+6 w test material-HFD (0.8 mg/g) (n=7). (K-M) Fasted blood glucose (K) and insulin (L) levels, and HOMA-IR (M; from K and L) at start, 9 w, and 15 w in hIAPPtg mice on HFD for 15 w (n=12) and in hIAPPtg mice at start, at 9 w on HFD, and at 9 w+6 w test material-HFD (0.8 mg/g) (n=7). Data are presented as mean±SEM, **P<0.01, **P<0.01, ***P<0.001 (Student's t test [A-D, I, and J]; paired 2-tailed t test.

FIG. 5 (A-E). Test material (“0304”) increases glucose uptake in skeletal muscle. (A) 2-deoxy-D-glucose (2-DG) uptake in rat skeletal L6 myotubes treated with test material as indicated (vehicle, n=8; 2.5 μM test material, n=6; 5.0 μM test material, n=6; and 10.0 μM test material, n=3). (B-D) Representative immunoblot analysis (B), quantification of AMPK expression (C) (n=6), and 2-DG glucose uptake (D) in siRNA transfected rat skeletal L6 myotubes treated with test material as indicated (n=8 for each condition). (E) [¹⁸F]-Fluorodeoxyglucose ([¹⁸F]-FDG) levels in calf and thigh muscle of CBA mice on high-fat diet (HFD) (n=8) or test material-HFD (2 mg/g) (n=6) for 2 w. Data are presented as mean±SEM, *P<0.05, ***P<0.001 (Student's t test).

FIG. 6 (A-H). Test material (“0304”) reduces amyloid formation in hIAPPtg diet-induced obese mice and improves arginine-induced insulin secretion in diet-induced obese mice. Representative images (A) and quantification (B) of Thio-S+ amyloid deposits in hIAPPtg mice on high-fat diet (HFD) for 16 w (n=9) and in mice on HFD for 9 w and then switched to test material-HFD (2 mg/g) for an additional 7 w (n=9). (C) Representative immunoblot analysis and quantification of test material stimulation of p-T172 AMPK in INS-1 insulinoma cells (vehicle, 2.5 μM and 5 μM test material, n=9; 10 μM test material, n=6, per condition, respectively), mouse primary islets (n=6 per condition), hIAPPtg mouse primary islets (n=6 per condition), and human islets (n=8 per condition). (D and E) Representative images (D) and quantification (E) of Thio-S+ amyloid deposits in hIAPPtg islets ex vivo cultured for 96 hours in 11 mM glucose (n=36 islets), 22 mM glucose (n=45 islets), and 22 mM glucose with 2.5 μM (n=42 islets), 5.0 μM (n=41 islets), and 10 μM test material (n=36 islets) as indicated (n=3 experiments for each). (F and G) Representative images (F) and quantification (G) of Thio-S+ amyloid deposits in hIAPPtg islets ex vivo cultured for 96 hours in 11 mM glucose (n=43 islets), 11 mM glucose with 5.0 μM 3-MA (n=59 islets), 22 mM glucose (n=54 islets), 22 mM glucose with 5.0 μM test material (35 islets), and 22 mM glucose with 5.0 μM test material and 5.0 μM 3-MA (n=44 islets) as indicated (n=3 experiments for each). (H) Plasma insulin profiles and AUC following i.p. injection of arginine (1 g/kg) in CBA mice fed a HFD (n=10) and test material-HFD (0.8 mg/g) (n=10) for 11 w. Data are presented as mean±SEM, *P<0.05, **P<0.01, ***P<0.001 (Student's t test).

FIG. 7 (A-H). Test material (“0304”) reverts established obesity at thermo-neutral conditions. (A and B) Body weight change over time (A) and food intake (B) in CBA mice switched between high-fat diet (HFD) (n=5) and test material-HFD (2 mg/g) (n=5) at housing and thermo-neutral conditions as indicated. (C-E) Oxygen consumption (VO2) (C), respiratory exchange ratio (RER) (D), and energy expenditure (EE) rates (E) in CBA mice on HFD (n=8) and test material-HFD (0.8 mg/g) (n=8) for 11 w. (F) Representative immunoblot analysis and quantification of ATGL and of p-S406 ATGL in inguinal white adipose tissue (iWAT) of CBA mice on HFD (n=10) and test material-HFD with 0.4 (n=5), 0.8 (n=10), and 2 mg/g (n=10) test material for 7 w. (G) Relative mRNA levels of Atgl, Cpt1b, Ppargc1a, and Cox8b in iWAT of 19 w-old CBA mice fed HFD (n=10) and test material-HFD and 2 mg/g test material (n=10) for 7 w. (H) Relative mRNA levels of Cd36, Fas, Scd1, Acc1, and Cpt1b in brown adipose tissue (BAT) of 19 w-old CBA mice fed HFD (n=10) and test material-HFD and 2 mg/g test material (n=10) for 7 w. Data are presented as mean±SEM, *P<0.05, **P<0.01, ***P<0.001 (Student's t test).

FIG. 8 (A-G). Test material (“0304”) reduces heart glycogen and improves stroke volume in diet-induced obese mice but does not cause cardiac hypertrophy. (A) Heart glycogen content in CBA mice fed high-fat diet (HFD) (n=10) and test material-HFD with 0.8 (n=10), and 2 mg/g (n=10) test material for 7 w. (B) [¹⁸F]-Fluorodeoxyglucose ([¹⁸F]-FDG) levels in heart of CBA mice fed HFD (n=8) or test material-HFD (2 mg/g) (n=6) for 2 w. (C) Heart weight in CBA mice fed HFD (n=10) and test material-HFD with 0.8 (n=10) and 2 mg/g (n=10) test material for 7 w. (D-F) End-diastolic volume (EDV) (D), end-systolic volume (ESV) (E), and stroke volume (SV) (F) in 16 w-old CBA mice fed regular diet (RD) (n=9) and in 18 w-old CBA mice fed a HFD (n=9) and test material-HFD with 0.8 mg/g test material (n=10) or 2 mg/g (n=10) test material for 6 w. (G) Heart rate (HR) in 16 w-old CBA mice fed RD (n=9), and 18 w-old CBA mice fed HFD (n=9) and test material-HFD with 0.8 (n=10) mg/g or 2 mg/g (n=10) test material for 6 w. Data are presented as mean±SEM, *P<0.05, **P<0.01, ***P<0.001 (Student's t test).

FIG. 9 (A-F). Test material (“0304”) improves microvascular blood flow and endurance in mice. (A and B) Representative laser Doppler image (A) and quantification (B) of peripheral blood fusion in left hind paw in vehicle—(n=10) and test material-treated (n=10) B6CBAF1/J (F1) mice on 8 w high-fat diet (HFD). (C and D) Endurance test (C) and lactate levels (D) after endurance test in vehicle—(n=14) and test material-treated (n=14) aged, lean B6 mice after 30 days of test material treatment. (E and F) Systolic (E) and diastolic (F) blood pressure in dogs single dosed with vehicle or test material at indicated concentrations. Data are presented as mean±SEM, *P<0.05, #P<0.05, **P<0.01, ##P<0.01, ***P<0.001, ###P<0.001 (Student's t test). In E and F, * refers to vehicle versus 540 mg/kg test material and # refers to vehicle versus 180 mg/kg test material.

FIG. 10 (A-E). Test material (“0304”) reduces fasting plasma glucose and blood pressure and increases microvascular perfusion in type 2 diabetes

(T2D) patients on Metformin. (A-C) Fasting plasma blood glucose (FPG) (A and B) and HOMA-IR (C) at day 1 and day 28 in placebo—(n=24) and test material-treated (n=25) T2D patients on Metformin with the FPG range >7 to <13.3 mmol/l (>126 to <240 mg/dl) at day 1. (D) Hyperemic microvascular perfusion assessed by dynamic T2*-quantification monitored by MRI at screening (MRI1) and at day 27-29 (MRI2) in calf muscle of the T2D patients. The test material group and the placebo group were split in half based on the time-to-peak (TTP) at baseline, where short TTP (placebo A [n=14], test material A [n=14]) and long TTP (placebo B [n=13], test material B [n=14]) represent a relative higher and lower rate of hyperemic perfusion, respectively. A significant shortening of TTP (P=0.043) and increase in Δ−T2* (P=0.034) was observed in subjects with relative lower rate of perfusion at baseline (long TTP) in the test material group (i.e., comparing test material B MRI1 with test material B MRI2) but not in subjects with short TTP, and there was no difference in subjects with either short or long TTP at baseline in the placebo group. (E) Absolute and relative change in systolic and diastolic blood pressure from day 1-28 in T2D patients on Metformin treated with placebo (n=27) or test material (n=30). Data are presented as mean±SEM, *P<0.05, **P<0.01, ***P<0.001 (Signed Wilcoxon's rank sum test).

FIG. 11. Combination Therapy with Compound 1 and Canagliflozin. Fasted blood glucose, fasted plasma insulin and HOMA-IR.

### EXAMPLES

The test material used in Examples 1 and 2 was 4-chloro-N-[2-[(4-chlorophenyl)methyl]-3-oxo-1,2,4-thiadiazol-5-yl]benzamide. This substance is referred to below as “the test material” and similar. The test material used in the study was synthesised and purified by Anthem Bioscience Pvt. Ltd. (Bangalore, India) for Baltic Bio AB (Umeå, Sweden) and Betagenon AB (Umeå, Sweden). 7.4.

### Example 1

We here describe the identification and testing of a PAN-AMPK activator, referred to as the test material, which was found to increase AMPK activity by suppressing the dephosphorylation of pAMPK.

Methods

Study Design

For animal experiments, no sample-size estimate was calculated before the study was executed. The experiments were not randomised unless otherwise stated. Investigators were not blinded to allocation during experiments and outcome assessment except during some measurements and quantifications (glucose tolerance test, glucose stimulated insulin secretion, arginine stimulation of insulin secretion, amyloid quantification, echocardiography, and ultrasound examination of the heart). For in vivo data, each n value corresponds to a single mouse. For amyloid quantification each n value corresponds to independent experiments and total number of islets investigated, respectively. For in vitro data, each n value corresponds to an independent experiment. If technical replicates were performed, then their mean was considered as n=1

For cell culture assays the test material was dissolved in DMSO Hybri-Max™ (Sigma, #D2650). For in vivo assays the test material was dissolved in 2% w/v methylcellulose, 4 mM phosphate buffer pH 7.4. Metformin (Sigma #D150959) was dissolved in in 2% w/v methylcellulose, 4 mM phosphate buffer pH.

Pharmacokinetics of the test material in C57BL/6JBomTac mice and NTac:SD rats were determined via UHPLC-ESI Triple Quad MSMS in plasma from non-fasted animals. The test material (40 mg/kg test material) was administered via oral gavage and 4-, 8-, 12- and 24-hours after administration blood was collected. Test material levels were determined in liver and brain from non-fasted Crl:CD (SD) rats administered the test material (40 mg/kg test material), once daily for 3 weeks, via oral gavage. The test material was extracted in acetonitrile and levels determined using UHPLC-ESI Triple Quad MSMS.

Animals

Female Crl:CD (SD) rats (Strain #001), male and female Wistar rats (Strain #003), and Zucker Crl:ZUC-Leprfa rats (Strain #185) were obtained from Charles River Lab. Female NTac:SD rats were obtained from Taconic. Male C57BL/6J (B6) mice were obtained from JAX mice (Jax #000664). Male C57BL/6JBomTac mice were obtained from Taconic (B6JBom). Male B6CBAF1/J (F1) mice were obtained from JAX mice (Jax #10011). CBA/CaCrl (CBA) mice were obtained from Charles River Lab (Charles River CBA/CaCrl). hIAPPtg mice were obtained from JAX mice (Jax #008232) and maintained by brother sister mating as well as by back-cross to CBA for more than 10 generations. Wild type littermates were used as controls for hIAPPtg mice.

14-15 weeks old male B6 were, based on starting weight, assigned into vehicle, Metformin, test material, and Metformin+test material treatment groups (100 mg/kg, orally once a day), 10 animals/group, and fed HFD throughout the 8 weeks experimental period.

7 weeks old male B6 were fed HFD for 7 weeks after which they, based on weight, were assigned into Test Material, and Metformin+Test Material treatment groups (100 mg/kg, orally once a day), and fed HFD for an additional 4 weeks.

12 weeks old CBA mice were randomised into a HFD and three test material-HFD groups (0.4 mg/g, 0.8 mg/g, and 2 mg/g) for 7 weeks. 16-17 weeks old CBA mice on regular diet was used as controls where indicated.

14 weeks old CBA mice were randomised into HFD and test material-HFD (2 mg/g) groups for 2 weeks while housed at 22° C. The two groups were then switched from HFD to test material-HFD and vice versa for an additional 4.5 weeks before transferred from 22° C. to 30° C. (thermoneutrality). After one week at 30° C. the diet was switched again and one week after the switch core body temperature were determined.

10-11 weeks old male hIAPPtg mice were randomised into vehicle and test material treatment groups (100 mg/kg, orally once a day) and fed HFD throughout the 6 weeks experimental period. 10-11 weeks old male hIAPPtg; CBA mice and wild type littermates were fed HFD for 9 weeks. After 9 weeks mice were either sacrificed or randomised into two groups either continuing on HFD or switched to test material-HFD (2 mg/g) for an additional 7 weeks.

8-10 weeks old Wistar male and female rats were treated by oral gavage with vehicle or test material at 100, 300 or 600 mg/kg/day for 6 months.

Animals were housed at 12:12 hour light/dark cycle in a temperature/humidity controlled (22° C./50% humidity) room and ad libitum feeding with either standard chow (Special Diet Service #801730), high fat diet (HFD) (Research diets, Inc. #D12492) or HFD (Research diets, Inc. #D12492) custom formulated with test material at 2 mg/g test material, 0.8 mg/g test material and 0.4 mg/g test material, respectively.

Cardiovascular Safety Pharmacology Study Using Radiotelemetry in Conscious Beagle Dogs Following a Single Oral Gavage

Telemetry analyses was performed by CiToxLAB North America (Laval, Quebec, Canada) in adult male beagle dogs, selected from CiToxLAB North America Dog Telemetry Colony, which had previously undergone surgery for telemetry transmitter implantation to monitor the arterial blood pressure, electrocardiogram, body temperature and locomotor activity (Data Science International, Model D70-PCT). All surgical procedures were performed in accordance with relevant Standard Operating Procedures. A telemetry transmitter was placed between the internal abdominal oblique muscle and the aponeurosis of the transverses abdominis of each animal. The pressure catheter was inserted into the femoral artery and the biopotential leads subcutaneously in a Lead II configuration. Test material was gavaged as a suspension at 60, 180, or 540 mg/kg.

Food Control, Body Weight and Composition

Food intake was measured weekly by giving each cage 200 g pellet. After one week, the amount of pellets consumed were calculated and adjusted according to the number of animals/cage. Body weight was measured weekly. Body composition was assessed using EchoMRI.

Echocardiography

Left ventricle structure and function were analysed with transthoracic, high-frequency echocardiography using the MS550D transducer. The examination was performed during light isoflurane anaesthesia (1.5-2.0% in 800 mL oxygen). Anaesthesia level was adjusted to keep the respiration rate at 80-110 breaths per minute. Left ventricular volumes were determined in B-mode using a Simpson's rule reconstruction. All images were analysed off-line in a blinded way using the Vevo LAB software 1.7.0. Stroke volume, cardiac output and heart rate were analysed, as well as wall thicknesses and left ventricle diameter. Three measurements/animal was performed for mean values.

Laser Doppler Imaging

9 weeks old F1 mice were fed HFD for 8 weeks and treated with either vehicle or test material (40 mg/kg, orally once a day). Veet hair removal cream was used to remove hair from the left hind limb one day prior to blood perfusion analysis. Mice were anaesthetised using isoflurane and placed on a heating pad. Blood perfusion was scanned using a PeriScan PIM II Images and LDPIwin software (version 2.6.1) was used to analyse the images.

Treadmill

For treadmill test 14 months old C57BL/6J mice with comparable running distance to exhaustion were assigned to two groups (14 animals/group) prior to treatment with either vehicle or test material (20 mg/kg, orally once a day) for 30 days. One week prior to the test mice went through a familiarization session of 5 minutes on the treadmill. Running protocol as follows: 15 minutes at 18.8 m/minute, 5 minutes at 24.4 m/minute and 27.1 m/minute until exhaustion. At exhaustion, blood lactate levels were measured using a lactate test meter (Arkray).

Indirect Calorimetry Measurements

21 weeks old CBA mice that had been on HFD or HFD formulated with 0.8 mg/g for 11 w were individually housed in the chamber with a 12-h light/12-h dark cycle in ambient temperature of 22° C. and allowed a minimum of 12 hours to acclimate to the chamber before data collection. VO2 and VCO2 rates were measured during 3 days by indirect calorimetry in TSE PhenoMaster Calorimetry metabolic cages (TSE Systems GmbH). The respiratory exchange ratio (RER) was calculated as a ratio of VCO2 produced/VO2 consumed. An RER of 0.7 indicates that fat is the predominant fuel source, while an RER closer to 1.0 indicates that carbohydrate is the primary fuel. Energy expenditure (EE) was calculated as the product of the calorific value (CV) of oxygen [=3.815+(1.232×RER)] and the volume of O2 consumed, i.e. [EE=CV×VO2 (kcal/h)] and related to lean weight.

Infrared Thermal Imaging

Skin temperature of non-sedated Zucker rats that had been treated with test material (10 mg/kg/day) or vehicle for 12 days was recorded with an infrared camera (FLIR ix series Extech IRC30, FLIR systems Inc.) and analysed with a specific software package (FLIR QuickReport version 1.2 SP2 (1.0.1.217). 9 rats per group was used and mean and maximum skin surface temperatures were measured for each animal 2 hours after final dose administration.

Glucose and Serum Related Measurements

Oral and intraperitoneal glucose tolerance tests combined with glucose stimulated insulin secretion were performed on 6 hours fasted non-sedated mice (Hypnorm (Veta Pharma)/Midazolam (Hamlenmice)) following i.p. injection of glucose (SIGMA #G7021) (0.75 g/kg body weight). Arginine-stimulated insulin secretion was determined following i.p. injection of arginine (SIGMA #A5131) (1 g/kg body weight) in non-fasted 21 weeks old CBA mice that had been on HFD or test material-HFD (0.8 mg/g) for 11 w. Blood glucose was measured using Glucometer (Ultra 2, One Touch) and plasma insulin analysed via the ultrasensitive mouse insulin ELISA kit (Chrystal Chem Inc. #90080). Area under the Curve (AUC) was calculated according to the trapezoid rule. The homeostasis model for insulin resistance (HOMA-IR) was calculated via: fasting blood glucose (mmol/L)×fasting plasma insulin (μU/L)/22.5. MATSUDA index was calculated via: [10000/sqrt (insulin (0 min)+glucose (0 min)+insulin mean (0-60 min)+glucose mean (0-60 min)]. Statistical significance was calculated via Student t-test (two-tailed).

Autophagic Flux Assay

INS-1E cells were incubated for 24 h with or without 5 μM test material in the presence or absence of 100 nM Bafilomycin A1 (InvivoGen #tlrl-baf1) during the last 60 min of incubation. Levels of LC3II were determined by Western blot analysis and quantified. Primary and secondary antibodies used are listed in Table 1.

TABLE 1
Antibodies

| | Antigen | Species | Supplier | Dilution-Condition |
|---|---|---|---|---|
| Primary | p-AMPKα | Rabbit | Cell Signaling | 1/500-3000 (depending on cell |
| antibodies | (Thr-172) | | (cat. nr. 2535) | line/tissue), TBST + 5% BSA |
| | panAMPKα | Rabbit | Cell Signaling | 1/2000-20,000 (depending on cell |
| | | | (cat. nr. 2532) | line/tissue), TBST + 5% BSA |
| | GAPDH | Rabbit | Cell Signaling | 1/50,000, TBST + 5% BSA |
| | | | (cat. nr. 2118) | |
| | p-ACC (Ser-79) | Rabbit | Cell Signaling | 1/500-3,000 (depending on cell |
| | | | (cat. nr. 3661) | line/tissue), TBST + 5% BSA |
| | ACC | Rabbit | Cell Signaling | 1/500-2,000 (depending on cell |
| | | | (cat. nr. 3662) | line/tissue), TBST + 5% BSA |
| | ATGL | Rabbit | Cell Signaling | 1/70,000, TBST + 5% BSA |
| | | | (cat. nr. 109251) | |
| | p-S406 ATGL | Rabbit | Cell Signaling | 1/2000, TBST + 5% BSA |
| | | | (cat. nr. 135093) | |
| | UCP-1 | Rabbit | Abcam (cat. | 1:100,000, TBST + 5% BSA |
| | | | Nr. 23841) | |
| | LC3B | Rabbit | Cell signaling | 1:500, TBST + 5% BSA |
| | | | (cat. nr. 2775) | |
| | β-Actin | Rabbit | Cell Signaling | 1/1000-5000 (depending on |
| | | | (cat. nr. 4967) | tissue), TBST + 5% BSA |
| secondary | peroxidase- | | Jackson | 1/10,000, TBST + 5% non-fat |
| antibodies | conjugated Affini- | | Laboratories, | dried milk |
| | pure Goat Anti- | | INC. (cat. nr. 111- | |
| | Rabbit IgG (H + L) | | 035-003) | |

Amyloid Analyses and Ex Vivo Islet Amy/Old Assay

Islets amyloid was quantification was done on pancreatic tissues isolated from hIAPPtg mice on HFD for 16 weeks (n=7 mice/n=69 islets) and hIAPPtg mice on HFD for 9 weeks then switched to test material-HFD (2 mg/g) for 7 weeks (n=5 mice/n=48 islets). Isolated pancreases was direct frozen, sectioned, and amyloid content quantified by staining with Thioflavin-S as previously described (Reference 2). For ex vivo analyses, islets were isolated by collagenase digestion of the pancreas (Reference 1) and cultured in RPMI medium 1640 (GIBCO #11879-0) supplemented with 11.1 or 22.2 mM glucose (GIBCO #A24940-01), 1% fetal bovine serum (GIBCO #10500), 50 U; μg/ml Pen/Strep (Gibco #15140-122), 10 mM Hepes (Umeå University, Laboratory medicine), 1 mM sodium pyruvate (GIBCO #11360-039) and 0.1% 2-Mercaptoethanol (Sigma #M3148). 0, 2.5, 5.0, and 10 μM test material were added from day 0 of culture. For assessing the effect of autophagy inhibition, 3-Methyladenine (3-MA, Aldrich #M9281), 5 μM, was added from day 0 of culture in combination with test material, 5 μM. The control contained DMSO 1:2000. Medium and compounds were changed every second day. After 92 hours of treatment islets were embedded, sectioned and amyloid content quantified by staining with Thioflavin-S as previously described (Reference 2). A minimum of 3 independent experiments was evaluated.

Determination of Cellular ATP Content

Wi-38 human lung fibroblast cells were stimulated with test material for 16 hours. Thereafter ATP content were determined with the ATP bioluminescence assay kit HS II (Roche Applied Science #11699709001) according to manufacturer's recommendations. The ATP data were normalised to cellular protein determined using BCA protein assay kit (Pierce #23225).

Western Blot Analysis

All cell lines were lysed in 0.1 M Tris-HCl, pH 6.8, 2% SDS, 10 mM sodium fluoride (SIGMA #S7920), 10 mM β-glycerophosphate (SIGMA #G6376), and 1 mM sodium vanadate (SIGMA #72060) and supernatant collected after 1 minute at 14,000 rpm. Islets (human and mouse) were lysed in 0.1 M Tris-HCl, pH 6.8, 2% SDS, protease inhibitor (Roche #04 693 124 001) and phosphatase inhibitor (Roche #04 906 837 001). Right calf muscle, heart, iWAT, and interscapular BAT was crushed in a pestle using liquid nitrogen and homogenised in ice cold RIPA buffer (150 mM sodium chloride (SIGMA #S7653), 1.0% NP40 (USB), 0.5% sodium deoxycholate (SIGMA #D6750), 0.1% SDS, 50 mM Tris pH 8.0, 20 mM sodium pyrophosphate (SIGMA #71515), 10 mM sodium fluoride, 10 mM β-glycerolphosphate, 1 mM sodium vanadate and protease inhibitor cocktail (Roche #04693124001), 1 tablet/10 ml lysis buffer). The supernatant was collected after 2 min at 14,000 rpm. This procedure was repeated until all fat was eliminated and the supernatant was clear, at 4° C. Samples were analysed on 4-15% polyacrylamide gels. Primary and secondary antibodies used are listed in Table 1. Values were normalised toward AMPKα, β-Actin, GAPDH or the respective non-phosphorylated counterpart.

qRT-PCR

For RNA purification calf muscle, iWAT, interscapular BAT, and left lateral liver lobe was crushed in a pestle using liquid nitrogen before turning to respective RNA kit. RNA from liver was prepared using Total RNA isolation Nucleospin II Kit (Macherey-Nagel #740955.50). RNA from adipose tissue was prepared using RNeasy Lipid Tissue Mini kit (Qiagen #74804). RNA from calf muscle tissue was prepared using RNeasy Fibrous Tissue Mini kit (Qiagen #74704). First strand cDNA synthesis was done using SuperScript III (First-Strand Synthesis SuperMix for qRT-PCR, Invitrogen #11752-250) according to the manufacturer's instructions. Total RNA was prepared from isolated islets using RNeasy Micro Kit (Qiagen #74004) and first strand cDNA synthesis was done using Superscript III (Invitrogen #18080-051) according to the manufacturer's instructions. Quantification of mRNA expression levels was performed essentially as previously described (Reference 3). Primers used for qRT-PCR are listed in Table 2. Tyrosine 3-monooxygenase/tryptophan 5-monoxygenase activation protein, zeta polypeptide (YWHAS) was used to normalise expression levels except for islets where TBP was used.

TABLE 2
Primers

| Exper- | | | |
|---|---|---|---|
| iment | Target | Forward primer | Reverse Primer |
| qRT- | Fas | TCCTGGAACGAGAACACGATCT | GAGACGTGTCACTCCTGGACT |
| PCR | Scd-1 | AGTGAGGCGAGCAACTGACTA | GGTGGTGGTGGTCGTGTAAGA |
| | Acc2 | CCCAGGAGGCTGCATTGAAC | ACGCGACGGTGAAATCTCTG |
| | Cpt1b | AGATCAAGCCGGTCATGGCA | TTGCCTGGGATGCGTGTAGT |
| | Glut1 | ATCCCAGCAGCAAGAAGG | CCAGTGTTATAGCCGAACTG |
| | TXNIP | ATCTTTATGTACGCCCCTGA | GGATCCACCTCAGTGTAAGT |
| | Atf4 | GGAATGGCCGGCTATGG | TCCCGGAAAAGGCATCCT |
| | Bip | TTCAGCCAATTATCAGCAAACTCT | TTTTCTGATGTATCCTCTTCACCAGT |
| | Pdia4 | TGACCCGGCCTACTTGCA | GTGTGGTGAAACTTGTAATCTTCTCTCA |
| | Edem2 | ACTTGGGAGAGACGCTGTGG | GGAGGTCCTTGATCGTGGCA |
| | Herpud1 | CATGTACCTGCACCACGTCG | GAGGACCACCATCATCCGG |
| | Dnajc3 | GACAGCTAGCCGACGCCTTA | GTCACCATCAACTGCAGCGT |
| | Tbp | GAATTGTACCGCAGCTTCAAAA | AGTGCAATGGTCTTTAGGTCAAGTT |
| | Ywhas | CTGCGTGACATCTGCAACGA | GGTTGCGAAGCATTGGGGAT |
| | Atgl | TCACCATCCGCTTGTTGGA | TGCTACCCGTCTGCTCTTTCA |
| | Cd36 | TCATATTGTGCTTGCAAATCCAA | GCTTTACCAAAGATGTAGCCAGTGT |
| | Acc1 | AGCCAGACATGCTGGATCTCAT | TGGGGATCTCTGGCTTACAGG |
| | Ppargc1α | CCGTAAATCTGCGGGATGATG | CAGTTTCGTTCGACCTGCGTAA |
| | Cox8b | GTTCACAGTGGTTCCCAAAG | AACGACTATGGCTGAGATCC |

Liver Lipid Extraction and Triglyceride Determination

0.2-0.3 g of liver was homogenised in 3 ml PBS before addition of 6 ml chloroform/methanol (2:1). Samples were mixed until phase separation no longer occurred and left at RT 30 minutes before centrifuged, 4,500 rpm, 5 minutes. The chloroform phase was transferred into pre-weighted glassware and kept at 4° C. O/N. Any water drops were removed and the chloroform evaporated by a stream of nitrogen before residual solvent was removed via SpeedVac, 15 minutes. The glassware was re-weighed, and total lipids calculated (mg/g liver). The residue was dissolved in 35% Triton X-100/methanol. Liver triglycerides were determined with a Serum Triglyceride Determination Kit (Sigma-Aldrich #TR0100). Analyses were done according to the manufacturer's recommendations with a minor modification for triglyceride determination, which was analysed at 560 nm instead of 540 nm.

Glycogen Determination

Heart glycogen content was determined using a Glycogen Assay Kit (Abcam #ab65620) according to the manufacturer's recommendations.

[1,2-¹⁴C] Acetate Incorporation into Total Lipids

Primary human hepatocytes (65,000-130,000 cells/well in 24-well dishes) were treated with vehicle control, 0.625, 1.25, 2.5 or 5 μM test material in serum free Williams' medium E, 2 hours, before addition of 0.25 μCi [1,2-¹⁴C]-acetate/well for an additional 4 hours. See Table 3 for growth conditions. 200 μl 0.5% trypsin was used to detach the cells before addition of 800 μl chloroform/methanol (2:1) and 500 μl 4 mM MgCl₂. The samples were vortexed and spun at 14,000 rpm, 2 minutes before discarding the aqueous layer. The procedure was repeated twice, first with 700 μl chloroform/methanol (2:1) and 500 μl 4 mM MgCl₂ and then 400 μl chloroform/methanol (2:1) and 500 μl 4 mM MgCl₂. The organic phase was transferred into a scintillation vial and evaporated to dryness by a stream of nitrogen. The residue was dissolved in 3 ml liquid scintillation cocktail (Optiphase HiSafe 3, Perkin Elmer #1200.437) and 14 C determined for 1 minute in a Wallac 1414 beta counter (Perkin Elmer). Before lipid extraction, 10 μl samples were used to determine protein concentration. 14 C-values were normalised to cellular protein concentration.

TABLE 3
Cell lines and islets

| | | Test material activation |
|---|---|---|
| Cell line/islets | Growth conditions | condition |
| Human Preadipocytes (Cell | Preadipocyte growth medium | Cells were treated (in the |
| Applications, Inc. #802h-05a) | (Cell Applications, Inc. #811- | presence of 0.1% DMSO) with |
| | 500) | 2.5, 5 or 10 μM test material in |
| | | serum free DMEM (Gibco |
| | | #21885) for 4.5 hours |
| Human skeletal muscle cells | Growth Medium (Cell | Cells were treated (in the |
| (Cell Applications, Inc. #150- | Applications, Inc. #151-500) | presence of 0.1% DMSO) with |
| 05a) | | 2.5, 5 or 10 μM test material in |
| | | serum free DMEM (Gibco |
| | | #21885) for 4 hours |
| Wi-38 human lung fibroblast | DMEM (Gibco #21885), | Cells were treated (in the |
| cells (LGC Promochem-ATCC | glucose 1 g/L, 10% FBS | presence of 0.1% DMSO) with |
| #CCL-75) | (Gibco #10500-064), 1 mM | 2.5, 5 or 10 μM test material in |
| | MEM NEAA (Gibco #11140- | serum free DMEM (Gibco |
| | 035), 25 μg/ml gentamicin | #21885) for 16 hours to |
| | (Gibco #15750) | analyze AMPK activation and |
| | | 16 hours to analyze ATP |
| | | content. |
| Human hepatocytes (Gibco | Resuspended and plated in | Cells were treated (in the |
| #HMCPMS) | Williams' medium E (Gibco | presence of 0.1% DMSO) with |
| | #A1217601) supplemented | 2.5, 5 or 10 μM test material in |
| | with hepatocyte plating | serum free Williams' medium |
| | supplement pack (Gibco | E (Gibco #A1217601) for 2 |
| | #CM3000). | hours (for western) or with |
| | | 0.625, 1.25, 2.5 or 5 μM test |
| | | material in serum free |
| | | Williams' medium E (Gibco |
| | | #A1217601) for 2 + 4 hours |
| | | ([1, 2-14C] acetate |
| | | incorporation) |
| Human umbilical vein | EBM (Lonza #CC-3121) | Cells were treated (in the |
| endothelial cells (Lonza #CC- | supplemented with EGM | presence of 0.1% DMSO) with |
| 2519) | singleQuot kit Suppl. and | 2.5, 5 or 10 μM test material in |
| | growth factor (Lonza #CC- | serum free EBM for 16 hours |
| | 4133) | |
| Insulinoma 1 (INS-1E) | RPMI medium 1640 (GIBCO | Cells were treated (in the |
| (AddexBio #00018009) | #21875-034), 11.1 mM | presence of 0.1% DMSO) with |
| | glucose (GIBCO #A24940-01), | 2.5, 5.0 and 10 μM test |
| | 10% fetal bovine serum | material in RPMI medium |
| | (GIBCO #10500), | (GIBCO #11879), 11.1 mM |
| | 1 mM sodium pyruvate | glucose (GIBCO #A24940-01), |
| | (GIBCO #11360-039), 10 mM | 1x MEM NEAA (Gibco |
| | Hepes (Umeå University, | #11140-050), 10 mM Hepes |
| | Laboratory medicine), 0.1% 2- | (Umeå University, Laboratory |
| | Mercaptoethanol (Sigma | medicine), 1 mM sodium |
| | #M3148), 50 U;μg/ml | pyruvate (GIBCO #11360- |
| | Pen;Strep (Gibco #15140- | 039), 0.1% 2-Mercaptoethanol |
| | 122), | (Sigma #M3148), 50 U; μg/ml |
| | | Pen;Strep (Gibco #15140- |
| | | 122), 1x N-2 (GIBCO #17502- |
| | | 048) for 2 hours |
| HeLa cells (kind gift from Prof. | DMEM (Gibco #21885), | Cells were treated (in the |
| Erik Lundgren, CMB, Umeå | glucose 1 g/L, 10% FBS | presence of 0.1% DMSO) with |
| University) | (Gibco #10500-064), 1 mM | 2.5, 5 or 10 μM test material in |
| | MEM NEAA (Gibco #11140- | serum free DMEM (Gibco |
| | 035), 25 μg/ml gentamicin | #21885) for 4 hours. 1 μM |
| | (Gibco #15750) | lonomycin were added the last |
| | | 20 minutes to control cells as |
| | | it activates AMPK. |
| Mouse islets | RPMI medium (GIBCO | Islets were treated (in the |
| | #11879), 1% fetal bovine | presence of 0.1% DMSO) with |
| | serum (GIBCO #10500), 11.1 | 2.5, 5.0 and 10 μM test |
| | mM glucose (GIBCO | material in serum free RPMI |
| | #A24940-01), 10 mM Hepes | medium supplemented as |
| | (Umeå University, Laboratory | described for growth |
| | medicine), 1 mM sodium | conditions for 2 hours |
| | pyruvate (GIBCO #11360- | |
| | 039), 0.1% 2-Mercaptoethanol | |
| | (Sigma #M3148), 50 U;μg/ml | |
| | Pen;Strep (Gibco #15140-122) | |
| Human islets | CMRL medium 1066 (GIBCO | Islets were treated (in the |
| | #21530-027), 10% fetal | presence of 0.1% DMSO) with |
| | bovine serum (GIBCO | 1.0, 2.5, 5.0 and 10 μM test |
| | #10500), 20 U;μg/ml Pen;Strep | material in CMRL medium |
| | (Gibco #15140-122) and 1X | 1066, serum free, |
| | GlutaMax (Gibco #35050-038) | supplemented as described |
| | | for growth conditions for 4 |
| | | hours |
| Rat L6 skeletal muscle cells | Dulbecco's Modified Eagle | N/A |
| (Cat No. CRL-1458, LGC | Medium (Gibco #31966) | |
| Promochem-ATCC) | | |

In Vivo Lipogenesis

15 weeks old CBA mice that had been on HFD or test material-HFD (2 mg/g) for 5 weeks were starved overnight and refed 90 minutes before injection with 1000 μCi 3H-NaOac (Perkin Elmer #NET003005MC) diluted in 0.9% NaCl. After 90 minutes 0.2-0.3 g liver were isolated and homogenised in 3 ml PBS before addition of 6 ml chloroform/methanol (2:1). Samples were mixed until phase separation no longer occurred and left at RT 30 minutes before centrifuged, 4,500 rpm, 5 minutes. The water phase was removed and 3 ml chloroform transferred to a scintillation vial and evaporated to dryness by a stream of nitrogen while standing in a 40° C. water bath. The residue was dissolved in 3 ml optiphase hisafe 3 (Perkin Elmer #1200.437) and ³H determined for 1 minute in a Wallac 1414 counter. ³H values were normalised to liver weight.

Glucose Uptake in L6 Myotubes

Rat L6 skeletal muscle cells grown in high-glucose (4.5 g/L) Dulbecco's Modified Eagle Medium (Gibco #31966), 10% fetal bovine serum (Gibco #10500-064) and 25 μg/ml gentamicin (Gibco 1 #5750) were induced to differentiate, by reducing the serum concentration to 2% for 14 days by which time the majority of myoblast had differentiated to myotubes. Myotubes were rinsed in serum-free low-glucose (1 g/L) DMEM (Gibco a #21885), treated with vehicle control, 2.5, 5 and 10 μM test material (serum-free low-glucose DMEM, 0.1% DMSO) for 2 hours, rinsed in serum-free DMEM w/o glucose (Gibco #11966) and thereafter incubated with the same for 20 minutes before addition of 1 μCi 2-Deoxy-D-glucose (2-DG) (Perkin Elmer #NET549A250UC) for 10 minutes. The cells were rinsed 3 times in serum-free DMEM w/o glucose and lysed in 1 ml RIPA buffer (150 mM Sodiumchloride, 1% NP40, 0.5% Sodiumdeoxycholate, 0.1% SDS, 50 mM Tris pH8.0). 300 μl were added to 4 ml liquid scintillation cocktail (Perkin Elmer #1200-437) before counted, 1 minute, in a Wallac 1414 beta counter. CPM was converted to arbitrary units by setting vehicle control as 1.

L6 myotubes were transfected with siAMPKα1 and α2 (Santa Cruz Biotechnology, Inc #sc-270142 and #sc-155985) or Silencer Negative Control siRNA (Ambion #AM4635) 6-7 days after starting differentiation, using lipofectamin RNAiMAX Transfection Reagent (Thermo Fisher Scientific #13778030) according to manufacturer's instructions (forward transfection). The final concentration of siRNA was set at 100 nM. The day before transfection the medium was changed to antibiotic-free medium (high-glucose, 4.5 g/L, Dulbecco's Modified Eagle Medium (Gibco #31966) and 2% fetal bovine serum (Gibco #10500-064). The level of AMPKα1 and α2 expression in cells transfected with siAMPKα1 and α2 and Silencer Negative Control siRNA, respectively was quantified by Western blot. Glucose uptake in the absence or presence of the test material (5 μM) for 4 h was assayed 72 hours after transfection as described above. Glucose uptake induced by the test material was normalised to that of vehicle control in cells transfected with siAMPKα1 and α2 and Silencer Negative Control siRNA, respectively.

In Vivo Glucose Uptake

12 weeks old CBA mice that had been on HFD or test material-HFD (2 mg/g) for 2 weeks were starved for 3 hours and then intravenously injected with 9±1.1 MBq of clinical grade 18F-Fluoro-Deoxy-Glucose ([¹⁸F]-FDG) (prepared at the Nuclear Medicine department at Norrlands University Hospital, Umeå) in saline in a total volume of 70-100 μL, during light isoflurane anaesthesia (1.5-2% in 800 mL/min 02). Mice were allowed to be awake and freely moving around in their cage after injection. After 180 minutes, mice were sacrificed under deep isoflurane anaesthesia and blood was removed by retrograde perfusion of PBS via the aorta. When the liver was pale, tissues were collected and scanned for a 10 minutes static uptake (nanoScan PET/CT, Mediso, Hungary). The tissues were then scanned ex vivo scanning to assess uptake in the isolated tissues. Images were reconstructed to a 0.4×0.4 mm resolution with a 3D iterative reconstruction with 4 iterations and 4 subsets (Mediso Tera-Tomo 3D), covering 98 mm axial distance, employing spike filter, delayed-window random correction, scatter and CT-based attenuation corrections. Volumes of interest were manually delineated over each tissue using imlook4d (www.dicom-port.com). Tracer uptake was quantified as standardised uptake values (SUV), using the formula: SUV=C/(l/m); with C being the measured tissue activity concentration (Bq/mL), l the injected dose (Bq), and m the body weight (g). C and l are decay corrected to the same time.

SAMS Peptide AMPK Activity Assay

50 ng AMPK (Upstate #14-305) was mixed in various combination with 2.5, 5 or 10 μM test material or 20 μM AMP (Sigma #A2002) in buffer (40 mM Hepes pH7.45, 0.5 mM DTT, 2 mM MgCl₂, 0.1% DMSO). In all settings 10 μg SAMS and 0.03 μCi/μl ³²P ATP (Perkin Elmer #NEG502Z500UC) were added. Total reaction volume was 25 μl, all components mixed on ice and the reaction carried out at 37° C., 15 minutes, before terminated with 5 μl phosphoric acid, and placed back on ice. 25 μl reaction were dried in on Whatman P81 filters, 50° C., 2 minutes, washed 3 times in 250 ml 1% phosphoric acid, 2 minutes, before added to 4 ml liquid scintillation cocktail (Perkin Elmer #1200-437) and counted, 1 minute, in a Wallac 1414 beta counter. The radioactivity correlates to enzyme activity.

AMPK Activation Assay

Table 3 contains origin of cell lines, growth conditions and settings for activation of AMPK via the test material. Human skeletal muscle cells were grown in growth medium obtained from the supplier of the cells until induction of myotube differentiation in DMEM (Gibco #21885) supplemented with 2% horse serum (Gibco #26050-070) for two days and thereafter treated with the test material as described in Table 3. Upon arrival, human hepatocytes were thawed for 1 minute at 37° C. before transferred into thawing medium (CHRM, Invitrogen #CM7000). After centrifugation, 10 minutes, 100× g at RT, the cell pellet was resuspended in Williams' medium E (Gibco #A1217601) supplemented with hepatocyte plating supplement pack (Gibco #CM3000). The cells were plated onto gelatin coated 60 mm dishes and then incubated overnight before treated with the test material as described in Table 3. INS-1E cells were pre-treated with medium for activation condition (Table 3) for 4 hours before addition of the test material. All cell lines were maintained in a humidified incubator at 37° C., 5% CO₂. Table 3 describes growth conditions and settings for activation of AMPK by the test material in mouse and human islets. After harvest mouse islets were cultured for two days in growth condition medium at 37° C., 5% CO₂ before treatment with the test material for 2 hours. Human islets from non-diabetic and T2D (type-2 diabetic) donors were provided through the JDRF award 31-2008-416 ECIT Islet for Basic Research program in compliance with Swedish law and the Ethical board for human research in Umea (www.epn.se). Upon arrival, the islets were transferred to 50 ml falcon tubes and left to settle for 5 min before removal of the supernatant and addition of culture medium (CMRL medium (GIBCO #21530-027), 10% fetal bovine serum (GIBCO #10500), 20 U/ml Pen:Strep (Gibco #15140-122) and 1× GlutaMax (Gibco #35050-038). Islets were washed with culture medium 3 additional times before transferred to Petri dishes and left to recover overnight in a humidified incubator at 37° C., 5% CO₂ before treatment with the test material for 4 hours

AMPK In Vitro De-Phosphorylation Assay

AMPKα2/β1/γ1 trimer (Life Technologies #PV4674, Lot 1261361B) (1 ng/μl) was incubated with 10 μM test material, 20 μM test material or 150 μM ADP (Sigma #A2754) +/−1 mM ATP (Sigma #A1852-1VL) in buffer (40 mM Hepes, 0.5 mM DTT, 0.2 mg/ml Gelatin (Sigma #G7041) and 0.4% DMSO), +/−PP2Ca (0.25-0.75 ng/μl) (Abcam ab51205-100; Lot GR54133-5) and 5 mM MnCl₂ (total volume 20 μl). AMPKα2/β1/γ1+/−ATP was preincubated with the test material and ADP for 2 minutes at 30° C. before addition of PP2C/MnCl₂ to start the dephosphorylation reaction which continued for 10-15 minutes at 30° C. Reactions were terminated by the addition of 0.17% BSA, 13 mM EDTA, 1.3×XT Sample buffer and 0.67% β-Mercaptoethanol in PBS. Samples were placed on ice, 5 minutes, heated at 100° C. for 5 minutes and chilled before run on a western gel. All steps were performed in high quality low-protein-binding eppendorf tubes. In a separate experiment 10 μM test material, 20 μM test material, 150 μM ADP alone or the combination of 10 μM test material+150 μM ADP and 20 μM test material+150 μM ADP was incubated with 1 ng/μl of AMPKα2/β1/γ1 or AMPKα1/β1/γ1 (Life Technologies #PV4672) trimer in buffer (40 mM Hepes, 0.5 mM DTT, 0.2 mg/ml gelatin and 0.4% DMSO)+/−0.25-0.5 ng/μl PP2Ca and 5 mM MnCl₂ or 5 mM MgCl₂. AMPK was sequentially preincubated with the test material and ADP or the combination for 2 minutes at 30° C. before sequential addition of PP2C/MnCl₂ or PP2C/MgCl₂ to start the dephosphorylation reaction which continued for 5-15 minutes at 30° C. The reaction was thereafter terminated and analysed as above.

PP2C Phosphatase Activity Assay

3 ng/μl PP2Ca (Abcam ab51205) and 5, 10 or 20 μM test material in buffer (50 mM Tris-HCL pH 7.5, 0.1 mM EDTA, 0.5 mM DTT, 5 mM MgCl₂) was used in the Sensolyte FDP protein phosphatase assay kit (Anaspec #71100) according to the manufacturer's instructions to measure the activity of PP2Ca. The fluorescence intensity was measured in a Bio Tek Synergy H4 multi-mode microplate reader.

Quantification and Statistical Analyses

Quantification of western blot experiments was performed using Image Lab (Bio-Rad Laboratories version 4.1 build 16) and Image-J Software (version 1.45s). Amyloid content quantification was performed using Image-J software (version 1.49m). All the statistical analyses of in vitro and mouse in vivo data were performed by two-tailed Students t-tests. We considered a value of P<0.05 to be statistically significant. Patient data analyses were performed using the mix model Anova test (throughout, 2-way ANOVA test was used for absolute changes and 1-way ANOVA test for percentage changes) and the non-parametric Wilcoxon Rank Sum test. The composite endpoint was analysed using Chi-Square, and Fisher's exact test.

Results

Test Material Suppresses Dephosphorylation of pAMPK In Vitro and Acts as a PAN-AMPK Activator in Cells.

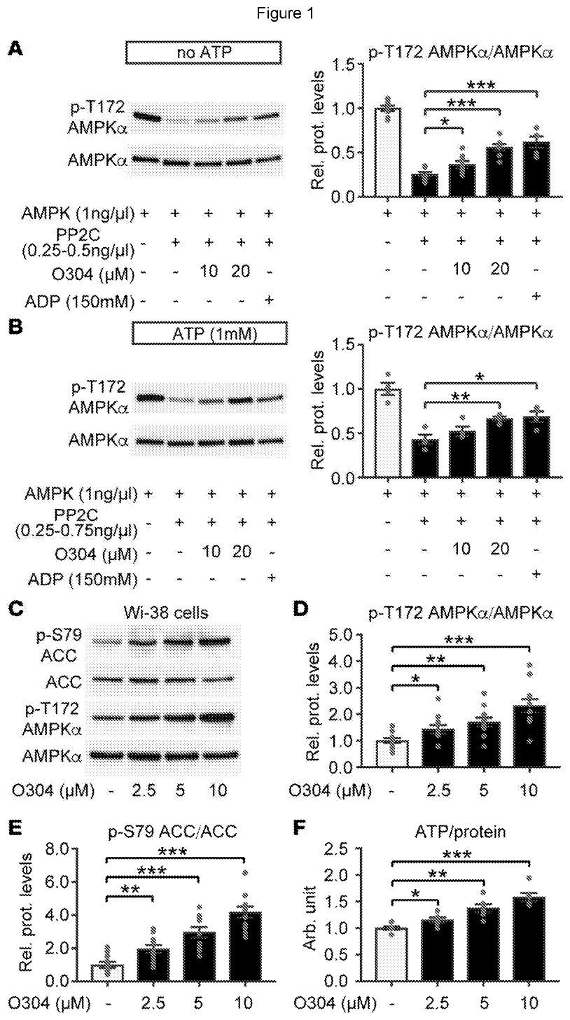

Consistently, in vitro test material suppressed protein phosphatase 2C—mediated (PP2C-mediated) dephosphorylation of p-T172 of human recombinant AMPKα, -β, and -γ trimers (FIG. 1A) without inhibiting the activity of PP2C. The test material also protected pAMPK from dephosphorylation in the presence of excess ATP (FIG. 1B) and acted in an additive manner with ADP, but it did not allosterically activate AMPK. Thus, the test material mimicked the effects of ADP, but not of AMP, on AMPK activity.

In nontransformed human Wi-38 lung fibroblast cells, the test material increased the levels of pAMPK, the downstream target p-S79 ACC (pACC), and the ATP/protein ratio in a dose-dependent manner (FIG. 1, C-F). Notably, the test material increased pAMPK in many different cell types containing a variety of different AMPK heterotrimers, which expressed either the (β1 or β2 subunit, including cells implicated in T2D, such as human skeletal myotubes and hepatocytes that preferentially express the β2 subunit. Thus, the test material acts as a PAN-AMPK activator in cells. The mechanism of action of the test material requires that cells express the major upstream kinase LKB1. Consistently, in HeLa cells, which are phenotypical LKB1 null, the test material failed to increase the very low basal levels of pAMPK and pACC, whereas as a control, the Ca2+ ionophore ionomycin, which activates AMPK via calcium/calmodulin-dependent protein kinase kinase (CaMKK), readily activated AMPK in these cells. Thus, the test material will only further increase AMPK activity in physiologically relevant cells with intrinsic AMPK activity.

Test Material Prevents Insulin Resistance and Dysglycemia in DIO Mice.

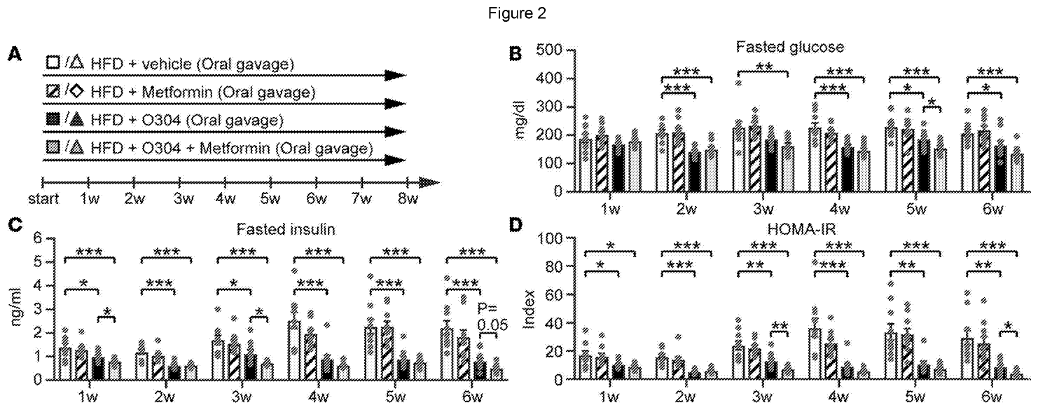

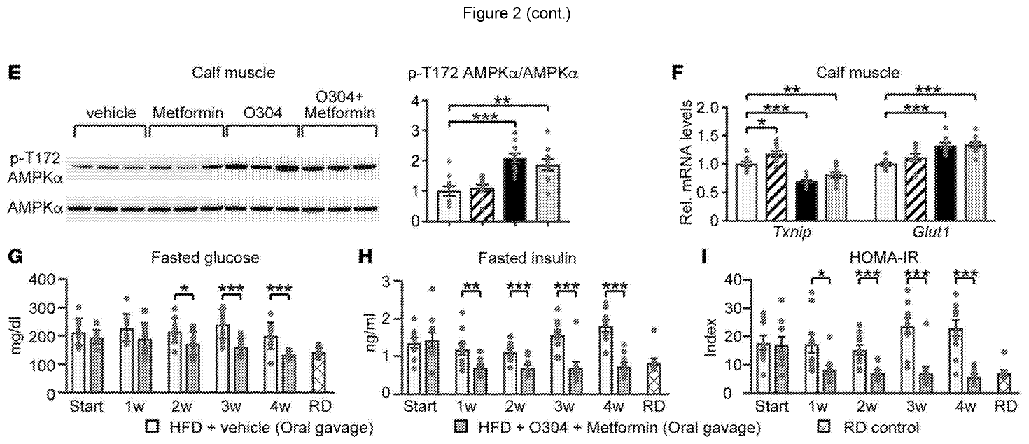

In rodents, the test material is orally available with a long plasma half-life but does not cross the blood-brain barrier. To address whether the test material alone or, as in the clinical setting, in combination with Metformin could mitigate dysglycemia and insulin resistance in vivo, mice were fed a high-fat diet (HFD), denoted “DIO” (diet-induced obesity) mice, and treated by oral gavage with vehicle, test material, Metformin, or test material+Metformin (100 mg/kg/day) each for 8 weeks (w) (FIG. 2A). With this regimen, test material and test material+Metformin, but not Metformin, averted the HFD-provoked increase in fasted glucose and plasma insulin levels (FIGS. 2, B and C). Consequently, compared with vehicle, test material and test material+Metformin-treated DIO mice did not develop insulin resistance as assessed by HOMA-IR calculations (FIG. 2D). Moreover, in line with the potent prevention of hyperglycemia, hyperinsulinemia, and insulin resistance, test material and test material+Metformin, but not Metformin, significantly increased pAMPK (FIG. 2E), reduced Txnip mRNA levels, and increased Glut1 mRNA levels (FIG. 2F) in calf muscle of DIO mice, which is consistent with both insulin-dependent and insulin-independent effects. In summary, the test material increased pAMPK in calf muscle and potently protected against hyperglycemia, hyperinsulinemia, and insulin resistance in DIO mice; Metformin showed no significant effect, whereas test material+Metformin appeared most effective and significantly reduced HOMA-IR compared with the test material alone.

In patients with T2D, the test material would be used in combination with Metformin to reduce established hyperglycemia. To mimic these conditions, mice were fed HFD for 7 w, which resulted in hyperglycemia and insulin resistance as compared with mice fed a regular diet (RD) (FIGS. 2, G-I), and were then treated with vehicle or test material+Metformin while continued on HFD for 4 w. Whereas a reduction in fasted insulin levels and HOMA-IR was evident after 1 w of treatment with test material+Metformin, fasted blood glucose levels were significantly reduced first after 2 w of treatment with test material+Metformin as compared with vehicle (FIG. 2, G-I). Prolonged treatment reduced blood glucose further and, after 4 w of treatment, fasted glucose levels were reduced to those of mice fed RD (FIG. 2G). Thus, the metabolic effects of test material+Metformin (i.e., reduction of hyperglycemia) largely resemble the effects of exercise and/or caloric restriction on hyperglycemia observed in man.

Test Material Prevents and Reverts Diabetes in hIAPPtg DIO Mice.

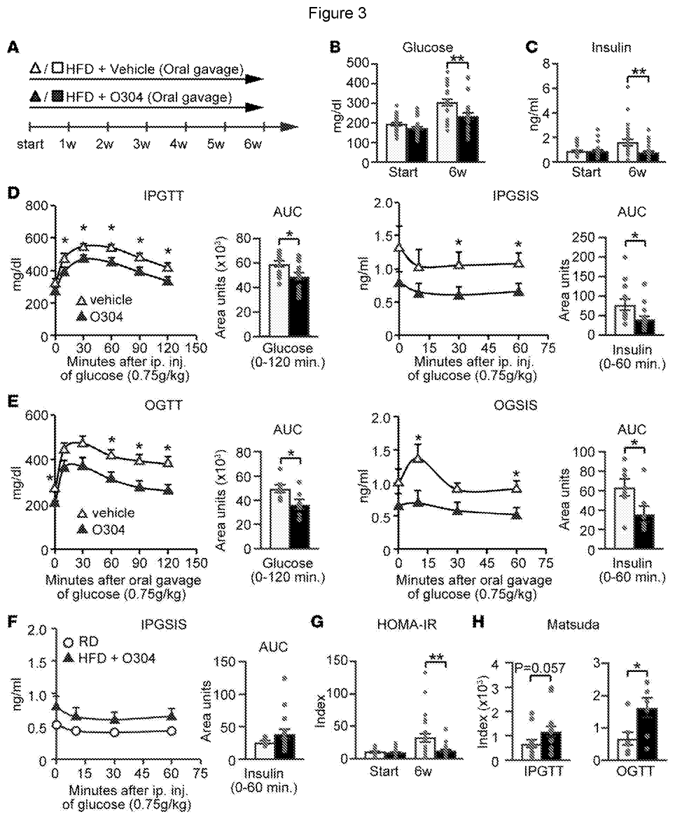

DIO mice become hyperglycemic but not overtly diabetic, and we therefore next explored the effect of the test material in a mouse model mimicking human T2D (i.e., HFD-induced insulin resistance/dysglycemia combined with 3 cell dysfunction). To this end, we used mice expressing the amyloidogenic human/APP (h/APP) gene under control of the rat insulin 2 promoter, denoted hIAPPtg mice, which were fed a HFD diet for 6 w (FIG. 3A). In hIAPPtg DIO mice, as compared with vehicle, test material gavaged at 100 mg/kg/day averted the increase in 6 h fasted blood glucose and plasma insulin levels (FIG. 3, B and C). I.p. (FIG. 3D) and oral (FIG. 3E) glucose-tolerance tests (GTTs) confirmed that the test material prevented the development of glucose intolerance and compensatory hyperinsulinemia, indicating a relative normalization of insulin hypersecretion that mirrored that of 10 w-old hIAPPtg mice on RD (FIG. 3F). Additionally, HOMA-IR and the Matsuda index model of whole-body insulin sensitivity showed that the test material suppressed the development of systemic insulin resistance in hIAPPtg DIO mice (FIG. 3, G and H).

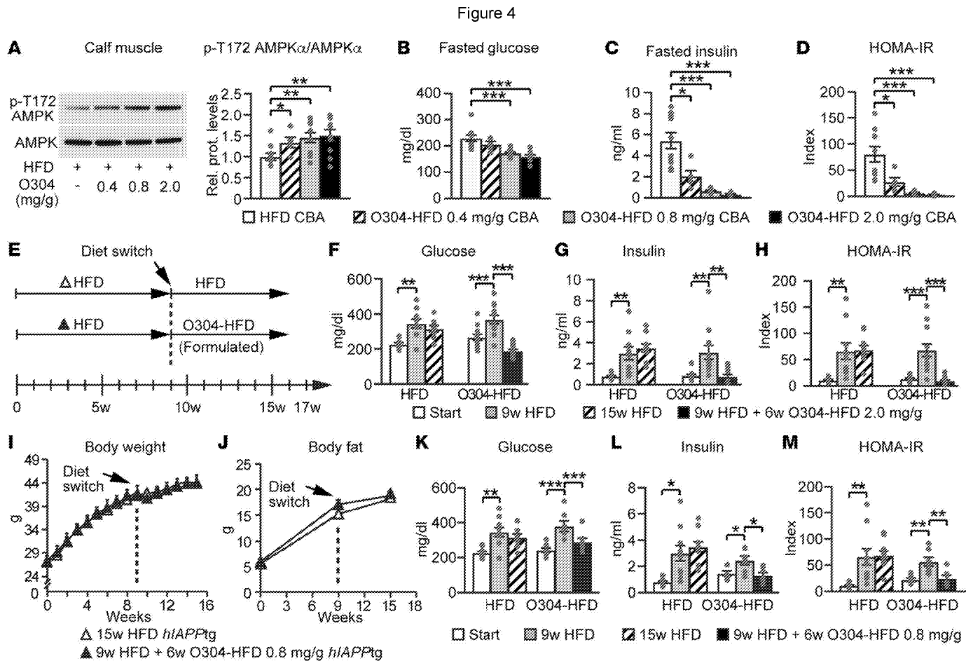

To test whether the test material could revert established diabetes and obesity in obese, diabetic hIAPPtg mice, and to avoid potential confounding effects of oral gavage on HFD-induced obesity, we formulated HFD with test material, denoted test material-HFD. To test the dose response effect of the test material on glucose homeostasis, we next fed CBA mice HFD or test material-HFD with 0.4, 0.8, and 2 mg/g of test material for 7 w. In CBA mice, test material-HFD dose-dependently increased pAMPK in calf muscle (FIG. 4A) and potently prevented dysglycemia, hyperinsulinemia, and insulin resistance (FIGS. 4, B-D). To test whether the test material could revert established diabetes, hIAPPtg mice were fed HFD for 9 w and then switched to test material-HFD (2 mg/g) for 7 w (FIG. 4E). At 6 w after the switch, test material-HFD had reverted established hyperglycemia, hyperinsulinemia, and insulin resistance—and, thus, diabetes (FIG. 4, F-H). Moreover, the test material induced body weight and body fat loss, despite increased food intake.

The potent effect of the test material on established diabetes and obesity in hIAPPtg mice on HFD formulated with 2 mg/g test material leaves open the possibility that the beneficial metabolic effects (FIG. 4, F-H) observed in these mice are secondary to the effects on weight and body fat. To address this issue, we therefore performed diet switch experiments on hIAPPtg mice using a lower test material-HFD concentration, where mice were fed HFD for 9 w and then either continued on HFD or switched to test material-HFD (0.8 mg/g) for 7 w. With this regimen, the switch to test material-HFD (0.8 mg/g) for 7 w did not provoke body weight or body fat loss (FIG. 4, I and J). Nonetheless, at 6 w after the switch to test material-HFD (0.8 mg/g), glucose and insulin levels—as well as HOMA-IR—were significantly reduced (FIG. 4, K-M), showing that, under these conditions, the beneficial metabolic effects of the test material are independent of any effect on weight and body fat loss. Together, these results show that the test material potently averts insulin resistance, hyperinsulinemia, hyperglycemia, and overt diabetes in a T2D mouse model of obesity-induced diabetes.

Test Material Increases Glucose Uptake in Skeletal Myotubes Ex Vivo and in Skeletal Muscle In Vivo.

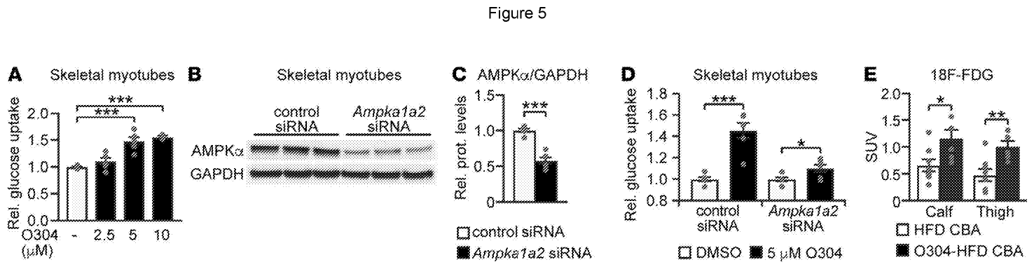

In skeletal muscle, AMPK activation has been implicated both in increasing insulin-independent glucose uptake and in reducing insulin resistance. Accordingly, in skeletal muscle myotubes, the test material increased 2-Deoxy-D-glucose (2-DG) uptake in a dose- and AMPK-dependent manner in the absence of insulin (FIGS. 5, A-D). Moreover, using PET analysis of tail vein injection of the radiolabeled glucose analog [¹⁸F]-Fluorodeoxyglucose ([¹⁸F]-FDG) a significant increase of [¹⁸F]-FDG uptake was observed in calf and thigh muscle of mice fed test material-HFD (2 mg/g) for 2 w compared with mice fed HFD (FIG. 5E), demonstrating that the test material promotes glucose uptake in skeletal muscle in vivo. Taken together, these findings provide evidence that the positive effects of the test material on glucose homeostasis is, at least in part, mediated by the test material stimulation of glucose uptake in skeletal muscle.

Test Material Reduces β Cell Stress and Promotes β Cell Rest.

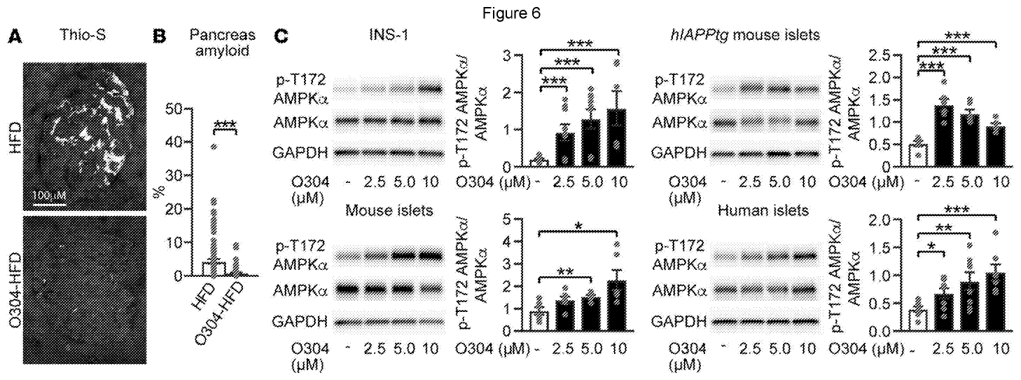

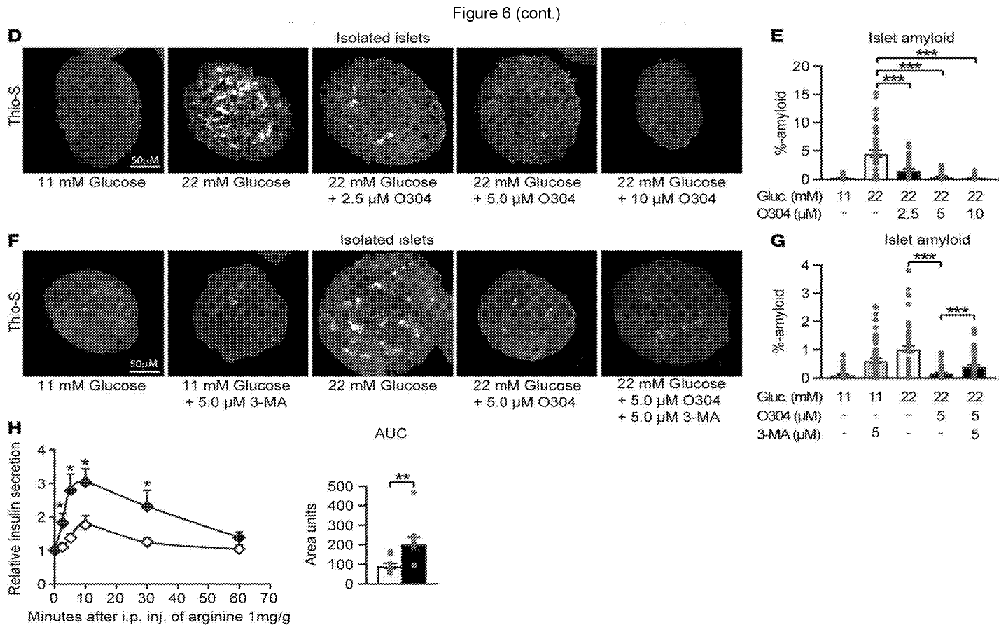

In T2D, toxic IAPP aggregates/amyloid is associated with β cell stress and β cell deterioration. In hIAPPtg HFD mice switched from HFD to test material-HFD (2 mg/g) for 7 w, the amount of islet amyloid formed was significantly reduced compared with that in mice continued on HFD for 7 w (FIGS. 6, A and B). The reduced amount of amyloid observed in test material-HFD—fed hIAPPtg mice may, however, be secondary to the amelioration of hyperglycemia and insulin resistance. Nonetheless, the test material directly increased pAMPKα in rat insulinoma INS-1 cells, isolated primary mouse WT and hIAPPtg islets, and human islets (FIG. 6C). To explore a potential direct effect of the test material on islet cells, we therefore next provoked amyloid formation by culturing isolated primary hIAPPtg islets at high glucose (22 mM) levels. the test material potently, in a dose-dependent manner, attenuated amyloid formation in hIAPPtg islets cultured at 22 mM glucose (FIGS. 6, D and E). Basal autophagy has been shown to protect β cells from hIAPP oligomer toxicity and AMPK activation promotes autophagy. Consistently, the test material enhanced autophagic flux in the β cell line INS-1E and in the presence of the autophagy inhibitor 3-MA; the preventive effect of the test material on amyloid formation at 22 mM glucose was significantly attenuated (FIGS. 6, F and G). AMPK activation has, however, also been shown to improve function and survival of metabolically stressed β cells through preservation of ER function, and the test material largely prevented an increased expression of unfolded protein response genes (i.e., indicative of ER stress) in primary mouse islets cultured at 22 mM glucose. Thus, the test material averts β cell amyloid formation in an obesity-induced T2D mouse model, as well as in isolated mouse islets cultured ex vivo at high glucose levels. Taken together, our findings suggest that the test material counteracts metabolically induced β cell stress and amyloid formation in vivo both by reducing hyperglycemia and systemic insulin resistance and by enhancing autophagy and/or ER function in β cells, although the exact mechanisms require further analyses.

The apparent ability of the test material to reduce β cell stress, likely both indirectly and directly, raises the question of whether the test material also promotes β cell rest that, in turn, would preserve long-term β cell function. Arginine stimulation of insulin secretion assesses first-phase insulin release (i.e., the ready releasable pool of granules) and provides an estimate of functional β cell reserve. To assess the effect of the test material on β cell function, we therefore next analysed arginine stimulation of insulin secretion. Arginine stimulation of insulin secretion was increased 2-fold in mice that had been fed test material-HFD (0.8 mg/g) for 11 w compared with that of mice fed HFD (FIG. 6H), providing further evidence that the test material mitigates β cell stress and promotes β cell rest, which in turn preserves/restores β cell function.

Test Material Reduces Obesity at Thermoneutral Conditions and Increases Energy Expenditure.

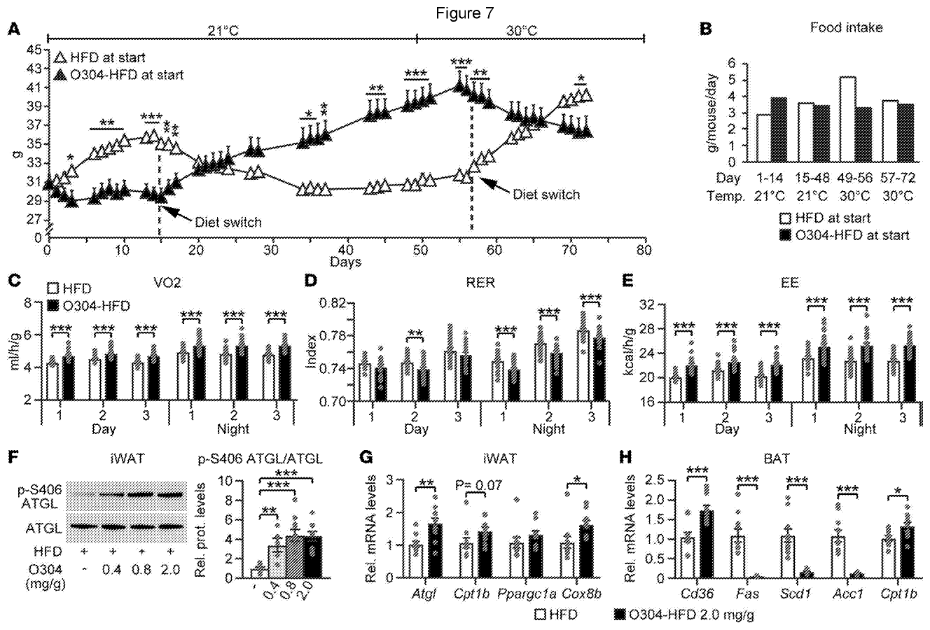

To further explore the effect of the test material on obesity, we performed crossover experiments. Mice fed HFD for 14 days rapidly gained weight, whereas those fed test material-HFD (2 mg/g) gained almost no weight, although they consumed more food than mice fed HFD during day 1-14 (FIG. 7, A and B). When HFD and test material-HFD were switched between these 2 groups of mice, mice that were switched from HFD to test material-HFD at day 15 rapidly started to lose weight, again with a relative increase in food intake; reciprocally, mice that switched from test material-HFD to HFD gained weight while reducing the relative food intake (FIGS. 7, A and B). We next tested whether the test material induced weight loss at thermoneutrality, and mice transferred from housing temperature to 30° C. from day 49 onwards while continued on test material-HFD still averted weight gain, whereas mice fed HFD continued to gain weight (FIG. 7A). Moreover, when mice housed at 30° C. switched diet from HFD to test material-HFD at day 57, they rapidly started to lose weight; reciprocally, mice that switched from test material-HFD to HFD started to rapidly gain weight (FIG. 7A). Under these conditions, only a small (0.2° C.) nonsignificant increase in core temperature was observed (37.7° C.±0.12° C. in HFD-, n=5, and 37.9° C.±0.08° C. in test material-HFD-treated mice, n=5). Thus, the test material also reduces obesity at thermoneutrality.

To directly address whether the test material averts obesity by increasing energy expenditure (EE), we measured oxygen consumption (VO2), respiratory exchange ratio (RER), and EE for 3 days in mice that had been fed HFD or test material-HFD (0.8 mg/g) for 11 w. VO2 was significantly increased during both light and dark periods in mice on test material-HFD compared with mice on HFD (FIG. 7C). RER was significantly decreased at day 2 during the light period and throughout the 3-day measurements during the dark period, providing evidence that mice fed test material-HFD switched their main energy source from carbohydrates to fatty acids (FAs) (FIG. 7D). As expected, EE was significantly increased during both light and dark periods (FIG. 7E). Taken together, these data strongly suggest that the test material suppresses weight gain by enhancing energy metabolism.

Test Material Increases ATGL Activity and Expression of Genes Associated with FA Oxidation in WAT and BAT.

In agreement with reduced body fat, test material-HFD—fed (2 mg/g) mice had markedly lower weights of inguinal white adipose tissue (iWAT) and epididymal WAT (eWAT) fat pads than HFD-fed mice. To reduce WAT depots, lipolysis needs to be enhanced. Desnutrin/Atgl, which encodes the rate-limiting enzyme catalysing basal triglyceride (TG) hydrolysis is a direct target of AMPK, and phosphorylation of S406 by AMPK increases ATGL activity, which should increase lipolysis. Accordingly, test material-HFD increased both p-S406 ATGL levels and Atgl mRNA levels in iWAT (FIG. 7, F and G). Moreover, Cpt1b, which increases mitochondrial FA uptake, and Cox8b, which would increase mitochondrial activity/FA oxidation, were also increased in iWAT of mice fed test material-HFD (2 mg/g) compared with mice fed HFD (FIG. 7G). The test material slightly, in a dose-dependent manner, reduced UCP1 expression in brown adipose tissue (BAT) as well as the low-level UCP1 expression in iWAT, arguing against ectopic expression of UCP1 in WAT as a mechanism for the antiobesity effect of the test material. Together, these data provide evidence that the test material, at least in part, averts obesity by increasing lipolysis and FA oxidation in WAT.

Activation of AMPK in BAT increases FA uptake, metabolic activity, and EE. Consistently, compared with HFD fed mice, weights of BAT pads were reduced in mice fed test material-HFD (2 mg/g), indicative of increased BAT metabolic activity. Notably, test material-HFD (2 mg/g) significantly increased the expression of Cd36, indicating enhanced FA uptake, as well as that of Cpt1b and potently reduced the expression of genes encoding FA synthase (Fas), Stearoyl-CoA desaturase 1 (Scd1), and Acc1 in BAT of DIO mice, which—in combination—should reduce de novo lipogenesis (DNL) and increase mitochondrial FA uptake/oxidation in BAT (FIG. 7H). Moreover, recent results provide evidence that heat can be produced in brown fat without intracellular lipolysis and that BAT can take up and burn FAs derived from lipolysis in WAT pads. Taken together, these findings leave open the possibility that an increase in both WAT and BAT activity in combination promote an increase in EE and reduced fat/body weight in test material-treated DIO mice.

Increased lipolytic flux from WAT to the liver may cause fatty liver. However, the test material dose-dependently suppressed lipid synthesis in human primary hepatocytes. The test material also reduced, by ˜45%, hepatic DNL; dose-dependently increased Cpt1b and decreased Acc2, Fas, and Scd1 mRNA levels in livers of DIO mice; and prevented and reduced hepatic steatosis in DIO mice.

Test Material Increases Cardiac pAMPK Levels, Increases Stroke Volume, and Reduces Cardiac Glycogen but does not Induce Cardiac Hypertrophy.

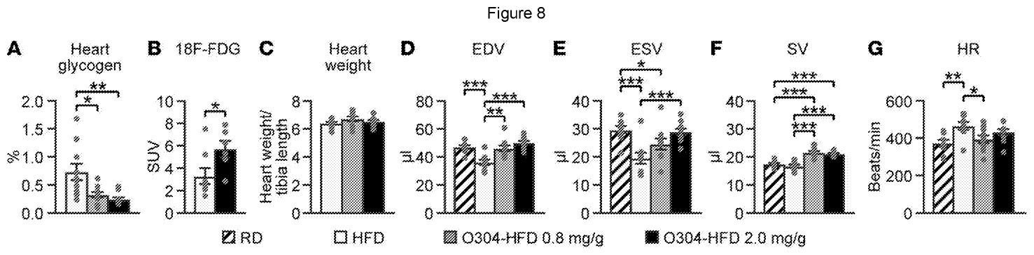

Exercise activates AMPK in the heart, increases glucose uptake, and reduces cardiac glycogen levels. Compared with mice fed HFD, mice fed test material-HFD at 0.8 or 2 mg/g for 7 w showed significantly increased pAMPK levels in the heart, and heart glycogen content was reduced in a dose-dependent manner (FIG. 8A). In a separate experiment, a significant increase of [¹⁸F]-FDG uptake was observed in the hearts of mice fed test material-HFD (2 mg/g) for 2 w compared with mice fed HFD (FIG. 8B). Thus, the cardiac effects of the test material resemble the cardiac effects of exercise. Compared with HFD, test material-HFD at 0.8 or 2 mg/g for 7 w did not cause an increase in heart weight/tibia length (FIG. 8C). Moreover, rats fed RD and gavaged for 6 months with the test material at 100, 300, and 600 mg/kg/day did not show increased heart/brain weight compared with vehicle. Therefore, test material-mediated AMPK activation in heart did not cause cardiac hypertrophy.

Exercise improves cardiac function by increasing stroke volume. We therefore examined the effects of the test material on LV function by echocardiography in mice fed RD, HFD, or test material-HFD at 0.8 mg/g or 2 mg/kg. Compared with RD, HFD caused a significant reduction in both end-diastolic volume and end-systolic volume and a small, but nonsignificant, decrease in stroke volume (FIGS. 8, D-F). test material-HFD normalized end-diastolic volume and dose-dependently improved, but did not fully restore, end-systolic volume (FIGS. 8, D and E). Importantly, test material-HFD 0.8 mg/g and 2 mg/g induced a significant increase (˜20%) in stroke volume compared with both RD and HFD (FIG. 8F). Notably, under these anesthetized conditions, HFD caused a significant increase in heart rate as compared with RD, whereas test material-HFD at 0.8 mg/g normalized and test material-HFD at 2 mg/g reduced heart rate (FIG. 8G). Thus, the test material normalized the HFD-induced decrease in end-diastolic volume and induced a significant increase in stroke volume, indicating that the test material mimics the beneficial effects of exercise on LV function.

Test Material Improves Microvascular Function and Endurance Capacity in Mice and Reduces Blood Pressure in Dogs.

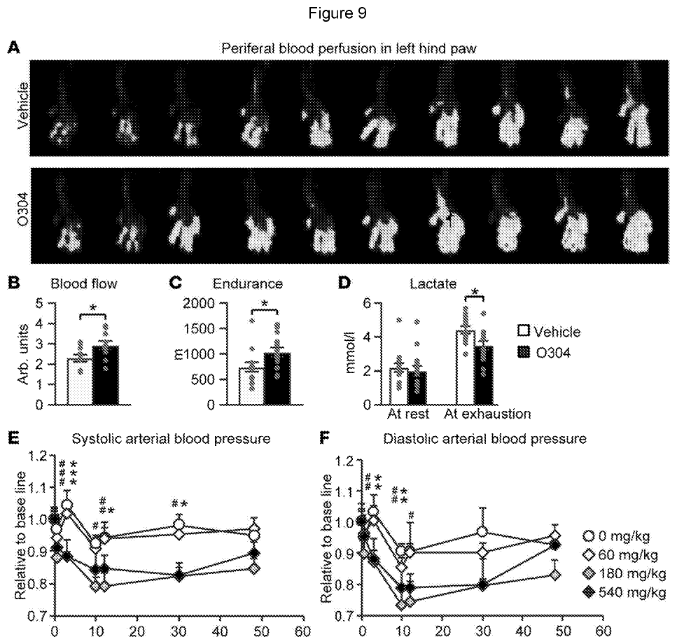

Reduced microvascular function and peripheral blood flow cause severe complications in T2D. AMPK activation in endothelial and smooth muscle cells promotes vasodilation, and to AMPK activator 5-aminoimidazole-4-carboxyamide-1-β-D-ribofuranoside (AICAR) increases microvascular perfusion in muscle. Thus, to elucidate a potential effect of the test material on peripheral blood flow, we used laser doppler imaging to monitor blood perfusion in left hind paws of DIO mice gavaged with vehicle or with test material (40 mg/kg/day) for 8 w. Under these conditions and without affecting body weight (mice on vehicle increased from 24.5-34 g and mice on test material from 25.6-36.5 g), test material—compared with vehicle—significantly increased microvascular blood flow in hind legs (FIGS. 9, A and B). In support of the notion that the test material increases microvascular blood flow, which would increase dissipation of heat, skin-surface temperature was increased in test material-treated Zucker rats.

Enhanced cardiovascular function is associated with improved endurance in humans and animals. To test whether the test material could improve endurance, we monitored running distance to exhaustion, and to avoid the confounding effect of varying degrees of obesity, we used weight-matched, 14-month-old mice fed RD and gavaged with vehicle or with test material (20 mg/kg/day) for 30 days. The treadmill exercise reduced body weights to a similar extent in mice on vehicle (from 33.4 to 31.4 g) and in mice on test material (from 34.1 to 32.2 g). Compared with vehicle, the test material significantly improved endurance capacity monitored as running distance to exhaustion (FIG. 9C), while significantly reducing the increase in blood lactate levels (FIG. 9D), indicating increased oxidative metabolism. Thus, consistent with the observed beneficial cardiovascular effects in DIO mice, the test material improves endurance capacity in lean sedentary aged mice fed RD.

The AMPK activator AICAR has been shown to acutely lower blood pressure and relax isolated resistance arteries of hypertensive rats. Thus, as part of the investigative new drug toxicological package, a telemetric study in conscious dog after single doses of the test material was conducted, and under these conditions, the test material acutely reduced blood pressure (FIGS. 9, E and F). Thus, the test material improves cardiac stroke volume, increases microvascular perfusion, and reduces blood pressure.

### SUMMARY

In DIO mice, the test material increased glucose uptake in skeletal muscle, reduced β cell stress, and promoted β cell rest. The test material improved peripheral microvascular perfusion and reduced blood pressure in animals. It also activated AMPK in the heart, increased cardiac glucose uptake, reduced cardiac glycogen levels, and improved LV stroke volume in mice, but it did not increase heart weight in mice or rats.

### Example 2—Phase IIa Clinical Trial

Methods

Clinical Study Design

An exploratory proof-of-concept randomised, parallel-group, double-blinded, placebo-controlled phase IIa 28-day study (TELLUS) of the first-in-class AMPK activator (the test material; 1,000 mg/day) was conducted in 65 T2D patients on Metformin for months, aiming at further exploring safety of test material and the effect of test material on FPG at a single-dose level.

TELLUS is listed in the EudraCT database protocol no. 2016-002183-13. The study was performed in accordance with ethical principles that have their origin in the Declaration of Helsinki and are consistent with International Conference of Harmonization (ICH)/Good Clinical Practice (GCP), European Union (EU) Clinical Trials Directive, and applicable local regulatory requirements. The study protocol was approved by the Regional Ethics Committee in Uppsala, Sweden, Project no/ID 0304-2016-02. Before performing any study-related procedures an informed consent form was signed and personally dated by all patients and by the Investigator.

Main inclusion criteria: Male and female patients, 18-80 years of age, with uncomplicated T2D, on stable T2D treatment with Metformin monotherapy for 3 months. HbA1c of ≥6.5% and 9.0%, and not FPG at day 1, was selected as the main inclusion criterion.

Main exclusion criteria: History of myocardial infarction (MI), unstable angina, stroke or transient ischemic attack (TIA). Congestive heart failure defined as New York Heart Association (NYHA) class III-IV. Any clinically significant abnormalities in physical examination, ECG or clinical chemistry results, as judged by the Investigator.

Clinical Study Compound

A good-manufacturing practice (GMP) batch of 5 kg test material was manufactured by Anthem BioSciences Pvt.Ltd, Bangalore, Karnataka, India. The suspension is composed of test material 20 mg/ml in 2% methylcellulose in phosphate buffer. A 2% methylcellulose suspension that colour matched the active product was used as placebo. The test material and placebo suspensions were manufactured, packaged and labelled by Recipharm Pharmaceutical Development AB, Solna, Sweden.

Clinical Methodology

Sixty-five (65) patients were randomised (1:1) to treatment with either the material or placebo. A screening visit (Visit 1) was performed within 3 weeks before randomisation and the start of IMP administration. Patients were randomised on Day 1 (Visit 2) and allocated to 28 days' treatment with either test material or placebo (1:1). Study visits to the clinic were performed 7, 14, 21, 28, 29 and 40 days (Visits 3 to 8) following randomisation and start of treatment. The patients were confined to the research clinic from the evening before Day 1 and Day 28 (Day −1 and Day 27, respectively) to ensure fasting conditions before samples for analyses of FPG were collected. Magnetic Resonance Imaging (MRI) scans after screening but before day 1 and after end of treatment were performed at the University Hospital in Uppsala, Sweden, according to standardised methods. Antaros Medical in Uppsala performed the data analysis. A clinical read of the acquired scans was performed by a radiologist at Antaros Medical. If clinically significant findings were noted by the radiologist, the Investigator was notified of the finding. The Investigator was to evaluate and handle the finding as per standard medical/clinical judgment.

Any findings were reported as either baseline events or adverse events, if they started, or worsened after administration of the first dose of IMP. The method used for assessing microvascular function in the calf muscle, (a proxy for oxygenation), include a dynamic MRI investigation of T2* determination before during and after reactive hyperemia (Reference 4). Sixty five (65) patients were randomised, 32 patients in the placebo group and 33 in the test material group, and 59 patients completed the study (28 and 31 in the two groups, respectively). HbA1c of 6.5% and 9.0%, and not FPG at day 1, was used as the inclusion criterion since MRI examinations had to be performed after screen but before day 1. Subsequently, a wide range of FPG values were observed at baseline, both <7 and >13.3 mmol/l, (<126 to >240 mg/dl) were 13.3 mmol/l (240 mg/dl) represents uncontrolled hyperglycemia, requiring a post hoc statistical analysis of change in FPG at day 28 compared to day 1 in T2D patients with FPG >7 mM and <13.3 mM at day 1.

Results

Test Material Improves Glucose Homeostasis in T2D Patients on Metformin.

Based on the beneficial metabolic and cardiovascular effects of the test material in preclinical species, the test material was selected for clinical development, and toxicological studies in rat and dog and a phase I safety clinical trial was successfully concluded. Thus, an exploratory 28-day proof-of-concept phase IIa clinical trial, denoted TELLUS, of the test material in 65 T2D patients stably on Metformin was performed. Apart from safety, FPG, insulin, and blood pressure were monitored, and microvascular perfusion in calf muscle was examined by MRI.

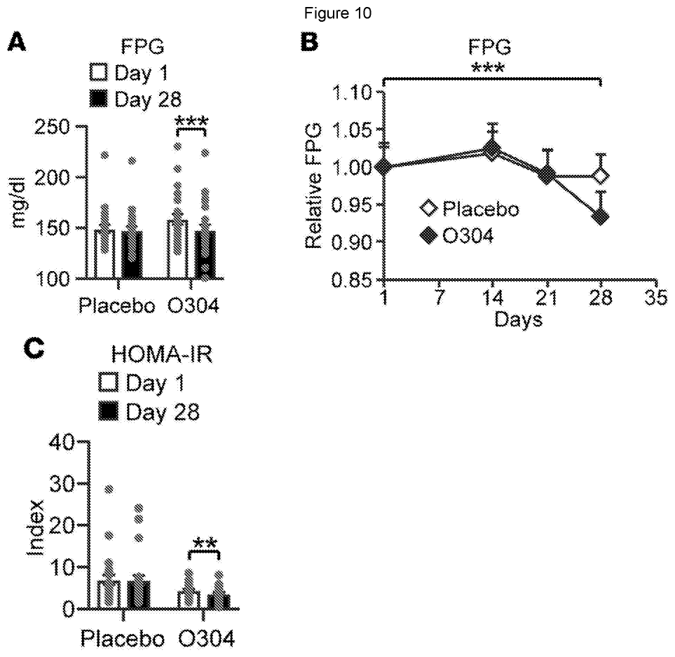

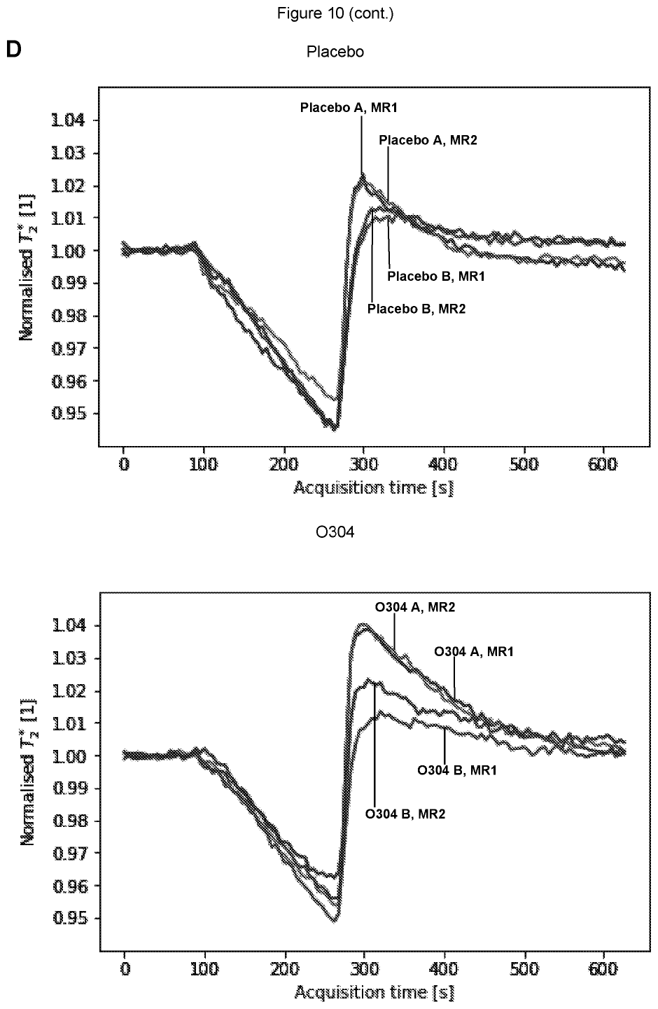

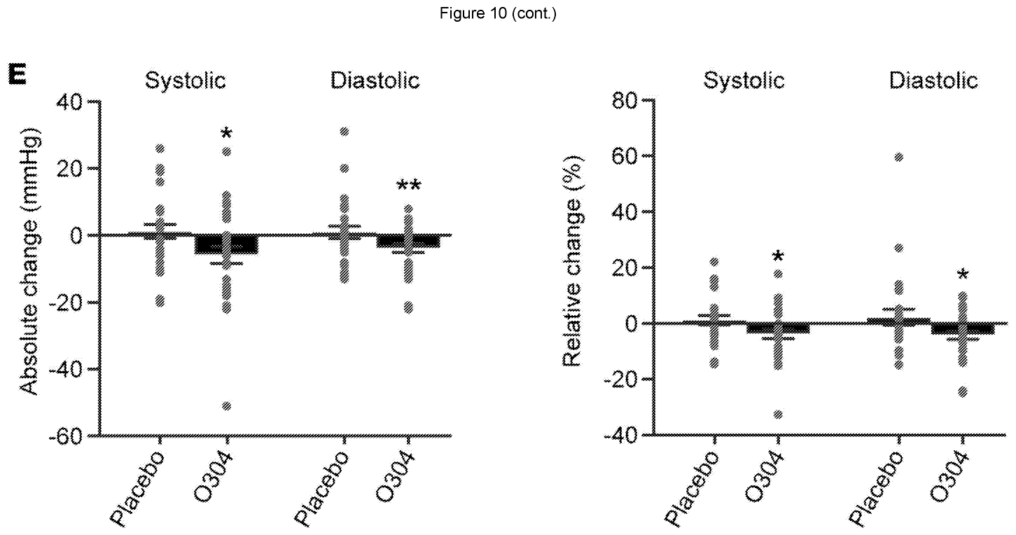

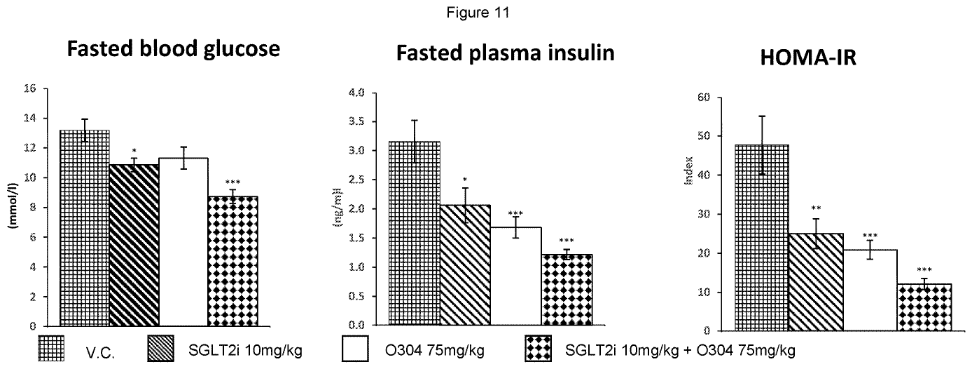

T2D patients needed to perform and pass MRI examinations before start of treatment to be included in the TELLUS study; therefore, HbA1c≥6.5% and ≤9.0% at screening, and not FPG at day 1, was used as inclusion criteria. Thus, a post hoc analysis was conducted of patients with FPG range >7 to <13.3 mmol/l, (>126 to <240 mg/dl) at day 1, where 13.3 mmol/l (240 mg/dl) represents uncontrolled hyperglycemia. The mean absolute reduction in FPG at day 28 compared with day 1 was −0.10 mM in the placebo group and −0.60 mM in the test material group (FIGS. 10, A and B). In the Wilcoxon's rank sum test there was a statistically significant absolute (P=0.010) and relative (P=0.018) reduction in FPG in the test material group compared with the placebo group, with P=0.049 for absolute change in the mix model ANOVA 2-way test and P=0.037 for relative change in the mix model ANOVA 1-way test. In the Wilcoxon test within the test material group, but not the placebo group, there was significant absolute (P=0.0002) (FIG. 10A) and relative (P=0.0003) reduction in FPG at day 28 compared with day 1. In DIO mice, a significant reduction in fasting blood glucose is observed after 2 w of treatment with test material+Metformin, and efficacy increases with duration of treatment (FIG. 2G). Thus, any effect of the test material on FPG in T2D patients would likely take at least 2 w to observe. Consistently, the significant reduction in FPG within in the test material group occurred between day 21 and day 28 (FIG. 10B), which is in accordance with the corresponding 14-day timeframe in DIO mice (FIG. 2G). Moreover, due to the long plasma t1/2 of the test material, the plasma steady-state concentration is not reached until day 14 in T2D patients. Notably, in the Wilcoxon test within the test material group, but not the placebo group, a statistically significant both absolute (P=0.0097) and relative (P=0.017) reduction in HOMA-IR were observed at day 28 compared with day 1 (FIG. 10C). Thus, the test material improved glucose homeostasis in T2D patients on Metformin.

Test Material Increases Peripheral Microvascular Perfusion in Calf Muscle of T2D Patients on Metformin.

Since T2D is associated with severe microvascular complications and the test material increased peripheral microvascular perfusion in mice, hyperemic microvascular perfusion was monitored in the TELLUS study by MRI and dynamic T2*-quantification (the time constant for transversal relaxation caused by local magnetic-field inhomogeneities) at screening and at days 27-29 in calf muscle of the T2D patients. The obtained time graphs of T2* values were analysed on an individual basis, and a set of parameters were extracted via automated curve fitting. As expected, when compared with the literature, the peripheral circulation status of the patients in the TELLUS study was at large not depressed at baseline, and a strong intervention effect signal could not be expected. Nevertheless, at day 28 compared with baseline in the 2-way ANOVA test, there was a statistically significant increase in Δ−T2* (P=0.026) in the test material group compared with the placebo group, defined as the difference between the minimum ischemic value and the peak hyperemic value, indicating increased hyperemic perfusion. Moreover, in the Wilcoxon test, there was a significant relative increase in the T2*-gradient (P=0.012), defined as the rate of increase of hyperemic perfusion in the test material group but not in the placebo group at day 28 compared with baseline. However, in subjects with comparably reduced peripheral circulation, peaks were poorly defined, making it difficult to correctly identify the peak properties following reactive hyperemia. Thus, in a post hoc analysis, curve fitting was instead performed at the group level, since image noise and signal drift was averaged over many subjects, and significance testing was performed by means of a permutation test, a nonparametric resampling technique. Under these conditions, compared with the placebo group, there was a significant increase in both Δ−T2* (P=0.037) and T2* gradient (P=0.024) at day 28 compared with baseline in the test material group. Thus, test material was found to increase microvascular perfusion in calf muscle of T2D patients on Metformin as assessed by changes in T2*-gradient and Δ−T2* at day 28 compared with baseline (Table 4).

TABLE 4

| | | | | | Hypothesis testing, P | | |
|---|---|---|---|---|---|---|---|
| | Test material | | Placebo | | Test material | Test material | Placebo |
| | MR1 | MR2 | MR1 | MR2 | vs. Placebo | MR1 vs. MR2 | MR1 vs. MR2 |
| T2*-grad (min⁻¹) | 0.528 | 0.665 | 0.458 | 0.401 | 0.024 | 0.035 | 0.327 |
| Δ-T2* [1] | 0.072 | 0.081 | 0.070 | 0.066 | 0.037 | 0.028 | 0.397 |

Study parameters assessed via curve-fitting of normalized group-averaged T2*-vs-time data for Test Material and placebo group. P-values are determined via permutation analysis and represent 2-sided tests. Grad, gradient.

Finally, to elucidate whether it was subjects with relatively lower perfusion at baseline that responded to treatment, the test material group and the placebo group were split in half based on the time-to-peak (TTP) at baseline, where short TTP and long TTP represent a relative higher and lower rate of hyperemic perfusion, respectively. MRI at baseline (MRI1) compared with MRI at end of treatment (MRI2) was then investigated with permutation analysis. From this stratified analysis, a significant shortening of TTP (P=0.043) and increase in Δ−T2* (P=0.034) was observed in subjects with a relative lower rate of perfusion at baseline (long TTP) in the test material group, but not in subjects with short TTP, and there was no difference in subjects with either short or long TTP at baseline in the placebo group (FIG. 10D). Thus, the test material preferentially increases hyperemic microvascular perfusion in calf muscle of T2D patients with a relative lower rate of perfusion at baseline.

Test Material Reduces Blood Pressure in T2D Patients on Metformin.

Microcirculation regulates peripheral vascular resistance, which—in combination with cardiac output—determines arterial blood pressure. AICAR acutely reduced blood pressure in spontaneously hypertensive rats, and the test material acutely reduced blood pressure in dogs (FIG. 9, E and F). Consistently, a mean absolute reduction in systolic (−5.8 mmHg) and in diastolic (−3.8 mmHg) blood pressure was observed at day 28 compared with day 1 in the test material group, whereas small increases of +1.2 mmHg and +0.9 mmHg, respectively, were observed in the placebo group. In the Wilcoxon test, within the test material group but not the placebo group, there was a statistically significant absolute reduction in both systolic (P=0.030) and diastolic (P=0.009) blood pressure and relative reduction in systolic (P=0.036) and diastolic (P=0.014) blood pressure (FIG. 10E). In the 1-way ANOVA test, there was a statistically significant relative reduction in systolic blood pressure (P=0.047) and diastolic blood pressure (P=0.044) in the test material group, compared with the placebo group. No significant change in mean heart rate was observed in either group at day 28 compared with baseline: placebo, −0.48; test material, −1.6 bpm. Thus, the test material reduces systolic and diastolic blood pressure in people with T2D. Hence, the effects of the test material on FPG, microvascular perfusion, and blood pressure translate from animals to T2D patients.

### SUMMARY

Consistently, in a 28-day proof-of-concept phase IIa clinical trial in T2D patients treated with Metformin, the test material reduced fasting plasma glucose (FPG) and homeostasis model assessment of insulin resistance (HOMA-IR), and it was well tolerated. The test material improved peripheral microvascular perfusion and reduced blood pressure in T2D patients. Thus, the major metabolic and vascular effects in animals translated to T2D patients.

### REFERENCES

1. Ahren B, Simonsson E, Scheurink A J, Mulder H, Myrsen U, and Sundler F. Dissociated insulinotropic sensitivity to glucose and carbachol in high-fat diet-induced insulin resistance in C57BL/6J mice. Metabolism. 1997; 46(1):97-106.
2. Ly P T, Cai F, and Song W. Detection of neuritic plaques in Alzheimer's disease mouse model. J Vis Exp. 201153).
3. Steneberg P, Rubins N, Bartoov-Shifman R, Walker M D, and Edlund H. The FFA receptor GPR40 links hyperinsulinemia, hepatic steatosis, and impaired glucose homeostasis in mouse. Cell Metab. 2005; 1(4):245-58.
4. Jacobi B, Bongartz G, Partovi S, Schulte A C, Aschwanden M, Lumsden A B, Davies M G, Loebe M, Noon G P, Karimi S, et al. Skeletal muscle BOLD MRI: from underlying physiological concepts to its usefulness in clinical conditions. J Magn Reson Imaging. 2012; 35(6):1253-65.

### Example 3—AMPK Activator+SGLT2 Inhibitor

Test Compound

The test materials used in this study were:

(A) 4-chloro-N-[2-[(4-chlorophenyl)methyl]-3-oxo-1,2,4-thiadiazol-5-yl]benzamide (referred to herein as “Compound 1”), synthesised and purified by Anthem Biosciences Pvt. Ltd. (Bangalore, India); and

(B) Canagliflozin.

Animals and Husbandry

Male C57BL/6J mice, 8 weeks of age were purchased from Jackson, Charles River Laboratories, Inc. (Germany). All animals were housed in the Umeå University animal facility (Umeå Centre for Comparative Biology; UCCB) with a 12:12 hour light-dark cycle (lights on at 6 a.m.) and a constant temperature of 21° C. The animals were ear marked with a unique identification number, and groups of 5 mice were housed in transparent polycarbonate cages that comply with the requirements of the Code of Practice for the housing and care of animals used in scientific procedures. Wood chips were used as bedding material and environmental enrichment was provided. Animals were allowed to acclimate for 15 weeks to their new environment before the onset of the study. The animals were allowed ad libitum access to tap water throughout the accommodation and study period. During the acclimation period, the animals were allowed a standard pelleted diet (CRM (E) Rodent, Special Diets Services, Scanbur BK, Sweden). At the start of the study, the standard diet was changed to a very high fat diet (vHFD; Cat. No. D12492, Research Diets, Inc.) and this diet was kept throughout the whole study period. All of the procedures were approved by the Local Ethics Review Committee on Animal Experiments, Umeå Region.

Reagents and Material for Biochemical Analysis

Ultra Sensitive Mouse insulin ELISA Kit (Cat. No. 90080, Crystal Chem.), OneTouch® Ultra® Test Strips (LifeScan, Inc.), OneTouch® Ultra®2 Blood Glucose Meter (LifeScan, Inc), Microvette® CB300 Potassium-EDTA vials (Cat. No. 16.444, Sarstedt).

Experimental Setup:

23 Weeks old male C57BL/6J mice were fed HFD to promote diet-induced obesity (DIO) and administered once daily by oral gavage; vehicle (phosphate buffer pH 7.3, 2% w/v methyl cellulose; n=11), canagliflozin 10 mg/kg (n=14), Compound 1 at 75 mg/kg (n=14) and the combination of canagliflozin 10 mg/kg and Compound 1 at da75 mg/kg (n=15). After 2 weeks of treatment, fasted (6 hour) blood glucose and plasma insulin levels (determined in tail vein blood samples) were analysed. Food intake and weight were monitored throughout the study.

Biochemical Analysis

Blood samples were collected from the tail vein in Potassium-EDTA vials and plasma was separated by centrifugation and stored at −20° C. until assayed. Plasma insulin was determined with mouse insulin ELISA (Ultra Sensitive Mouse insulin ELISA Kit). Glucose concentrations were analysed in tail vein blood using a OneTouch® Ultra®2 Blood Glucose Meter (LifeScan, Inc).

Data Analysis

Results shown in the figures are expressed as means±standard error of the mean (S.E.M.) for the number of animals per group. Statistical significance between the control group and the three treatment groups were analysed by Student's t-test, with P<0.05 considered statistically significant.

Results

The results are shown in FIG. 11. The combination of Compound 1 and a SGLT2 inhibitor provided a statistically significant (***) reduction in the fasted blood glucose, fasted plasma insulin and HOMA-IR results. Compound 1 alone showed a statistically significant (***) reduction in the fasted plasma insulin and HOMA-IR results. The SGLT2 inhibitor resulted in a lesser reduction in the fasted blood glucose, fasted plasma insulin and HOMA-IR results.

### CONCLUSIONS

SGLT2 inhibitors fail to show anti-glycaemic efficacy in T2D patients with impaired renal function and are therefore contra-indicated in this group of patients. However, Compound 1 and canagliflozin in combination has been found to potently and synergistically reduce hyperglycaemia, hyperinsulinemia and insulin resistance in diet-induced obese mice (FIG. 11), indicating that the combination of these two classes of compounds may both improve glucose homeostasis and prevent diabetic kidney disease in T2D patients in a potent manner. The subgroup of T2D patients having severe insulin-resistant diabetes (who are obese (BMI ˜35), insulin resistant and hyperinsulinaemic) and have a fivefold higher risk of developing diabetic kidney disease and currently lack efficient treatment. These patients may in particular benefit from such a combination therapy of the compound of formula I and a SGLT2 inhibitor.

1. A method of treating diabetes, said method comprising administering a compound of formula I, 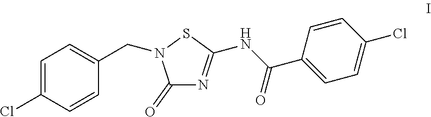 [SMILES: O=C(Nc1nc(=O)n(Cc2ccc(Cl)cc2)s1)c1ccc(Cl)cc1]
or a pharmaceutically acceptable salt, solvate or prodrug thereof, to a subject in need thereof, wherein the subject is identified as having severe insulin-resistant diabetes.

2. The method according to claim 1, which is a method of treating type 2 diabetes.

3. The method according to claim 1, wherein the subject is obese.

4. The method according to claim 3, wherein the subject has a BMI of at least 30 kg/m².

5. The method according to claim 1, wherein the subject has a blood C-peptide concentration of at least 1.4 nmol/L.

6. The method according to claim 1, wherein the subject has an increased risk of susceptibility to diabetic kidney disease.

7. The method according to claim 1, wherein the subject has diabetic kidney disease.

8. The method according to claim 1, wherein the bodyweight of the subject is reduced.

9. The method according to claim 1, wherein the subject's renal hemodynamics are improved.

10. The method according to claim 1, wherein the subject is human.

11. The method according to claim 1, wherein the compound of formula I, or pharmaceutically acceptable salt, solvate or prodrug thereof, is administered orally, nasally, parenterally or by inhalation.

12. The method according to claim 1, wherein the compound of formula I, or pharmaceutically acceptable salt, solvate or prodrug thereof, is administered to a subject at a daily dose in the range of from about 1 to about 2000 mg.

13. A combination of:
(A) compound of formula I as defined in claim 1, or a pharmaceutically acceptable salt, solvate or prodrug thereof; and
(B) a sodium-glucose transport protein 2 (SGLT2) inhibitor, or a pharmaceutically acceptable salt, solvate or prodrug thereof.

14. A pharmaceutical formulation comprising a combination as defined in claim 13 in admixture with a pharmaceutically acceptable adjuvant, diluent or carrier.

15. A kit-of-parts comprising:
(A) a composition comprising a compound of formula I as defined in claim 1, or a pharmaceutically acceptable salt, solvate or prodrug thereof, and a pharmaceutically acceptable adjuvant, diluent or carrier; and
(B) a composition comprising a sodium-glucose transport protein 2 (SGLT2) inhibitor, or a pharmaceutically acceptable salt, solvate or prodrug thereof, and a pharmaceutically acceptable adjuvant, diluent or carrier;
which components (A) and (B) are each provided in a form that is suitable for administration in conjunction with the other.

16. (canceled)

17. The method according to claim 1, wherein a sodium-glucose transport protein 2 (SGLT2) inhibitor, or a pharmaceutically acceptable salt, solvate or prodrug thereof is also administered to the subject.

18. The method according to claim 17, wherein the compound of formula I and the sodium-glucose transport protein 2 (SGLT2) inhibitor are administered sequentially, separately and/or simultaneously to the subject.

19. The combination according to claim 13, wherein the sodium-glucose transport protein 2 (SGLT2) inhibitor is selected from the group consisting of canagliflozin, dapagliflozin, empagliflozin, ipragliflozin, tofogliflozin, sergliflozin etabonate, remogliflozin etabonate, ertugliflozin and sotagliflozin, and pharmaceutically acceptable salts, solvates and prodrugs thereof.

20-22. (canceled)

23. The pharmaceutical formulation according to claim 14, wherein the sodium-glucose transport protein 2 (SGLT2) inhibitor is selected from the group consisting of canagliflozin, dapagliflozin, empagliflozin, ipragliflozin, tofogliflozin, sergliflozin etabonate, remogliflozin etabonate, ertugliflozin and sotagliflozin, and pharmaceutically acceptable salts, solvates and prodrugs thereof.

24. The kit-of-parts according to claim 15, wherein the sodium-glucose transport protein 2 (SGLT2) inhibitor is selected from the group consisting of canagliflozin, dapagliflozin, empagliflozin, ipragliflozin, tofogliflozin, sergliflozin etabonate, remogliflozin etabonate, ertugliflozin and sotagliflozin, and pharmaceutically acceptable salts, solvates and prodrugs thereof.


SEQUENCE LISTING (38 sequences)

SEQ ID NO: 1 - DNA, 22 bp, Artificial Sequence
  Fas forward primer
tcctggaacgagaacacgatct

SEQ ID NO: 2 - DNA, 21 bp, Artificial Sequence
  Fas reverse primer
gagacgtgtcactcctggact

SEQ ID NO: 3 - DNA, 21 bp, Artificial Sequence
  Scd-1 forward primer
agtgaggcgagcaactgacta

SEQ ID NO: 4 - DNA, 21 bp, Artificial Sequence
  Scd-1 reverse primer
ggtggtggtggtcgtgtaaga

SEQ ID NO: 5 - DNA, 20 bp, Artificial Sequence
  Acc2 forward primer
cccaggaggctgcattgaac

SEQ ID NO: 6 - DNA, 20 bp, Artificial Sequence
  Acc2 reverse primer
acgcgacggtgaaatctctg

SEQ ID NO: 7 - DNA, 20 bp, Artificial Sequence
  Cpt1b forward primer
agatcaagccggtcatggca

SEQ ID NO: 8 - DNA, 20 bp, Artificial Sequence
  Cpt1b reverse primer
ttgcctgggatgcgtgtagt

SEQ ID NO: 9 - DNA, 18 bp, Artificial Sequence
  Glut1 forward primer
atcccagcagcaagaagg

SEQ ID NO: 10 - DNA, 20 bp, Artificial Sequence
  Glut1 reverse primer
ccagtgttatagccgaactg

SEQ ID NO: 11 - DNA, 20 bp, Artificial Sequence
  TXNIP forward primer
atctttatgtacgcccctga

SEQ ID NO: 12 - DNA, 20 bp, Artificial Sequence
  TXNIP reverse primer
ggatccacctcagtgtaagt

SEQ ID NO: 13 - DNA, 17 bp, Artificial Sequence
  Atf4 forward primer
ggaatggccggctatgg

SEQ ID NO: 14 - DNA, 18 bp, Artificial Sequence
  Atf4 reverse primer
tcccggaaaaggcatcct

SEQ ID NO: 15 - DNA, 24 bp, Artificial Sequence
  Bip forward primer
ttcagccaattatcagcaaactct

SEQ ID NO: 16 - DNA, 26 bp, Artificial Sequence
  Bip reverse primer
ttttctgatgtatcctcttcaccagt

SEQ ID NO: 17 - DNA, 18 bp, Artificial Sequence
  Pdia4 forward primer
tgacccggcctacttgca

SEQ ID NO: 18 - DNA, 28 bp, Artificial Sequence
  Pdia4 reverse primer
gtgtggtgaaacttgtaatcttctctca

SEQ ID NO: 19 - DNA, 20 bp, Artificial Sequence
  Edem2 forward primer
acttgggagagacgctgtgg

SEQ ID NO: 20 - DNA, 20 bp, Artificial Sequence
  Edem2 reverse primer
ggaggtccttgatcgtggca

SEQ ID NO: 21 - DNA, 20 bp, Artificial Sequence
  Herpud1 forward primer
catgtacctgcaccacgtcg

SEQ ID NO: 22 - DNA, 19 bp, Artificial Sequence
  Herpud1 reverse primer
gaggaccaccatcatccgg

SEQ ID NO: 23 - DNA, 20 bp, Artificial Sequence
  Dnajc3 forward primer
gacagctagccgacgcctta

SEQ ID NO: 24 - DNA, 20 bp, Artificial Sequence
  Dnajc3 reverse primer
gtcaccatcaactgcagcgt

SEQ ID NO: 25 - DNA, 22 bp, Artificial Sequence
  Tbp forward primer
gaattgtaccgcagcttcaaaa

SEQ ID NO: 26 - DNA, 25 bp, Artificial Sequence
  Tbp reverse primer
agtgcaatggtctttaggtcaagtt

SEQ ID NO: 27 - DNA, 20 bp, Artificial Sequence
  Ywhas forward primer
ctgcgtgacatctgcaacga

SEQ ID NO: 28 - DNA, 20 bp, Artificial Sequence
  Ywhas reverse primer
ggttgcgaagcattggggat

SEQ ID NO: 29 - DNA, 19 bp, Artificial Sequence
  Atgl forward primer
tcaccatccgcttgttgga

SEQ ID NO: 30 - DNA, 21 bp, Artificial Sequence
  Atgl reverse primer
tgctacccgtctgctctttca

SEQ ID NO: 31 - DNA, 23 bp, Artificial Sequence
  Cd36 forward primer
tcatattgtgcttgcaaatccaa

SEQ ID NO: 32 - DNA, 25 bp, Artificial Sequence
  Cd36 reverse primer
gctttaccaaagatgtagccagtgt

SEQ ID NO: 33 - DNA, 22 bp, Artificial Sequence
  Acc1 forward primer
agccagacatgctggatctcat

SEQ ID NO: 34 - DNA, 21 bp, Artificial Sequence
  Acc1 reverse primer
tggggatctctggcttacagg

SEQ ID NO: 35 - DNA, 21 bp, Artificial Sequence
  Ppargc1alpha forward primer
ccgtaaatctgcgggatgatg

SEQ ID NO: 36 - DNA, 22 bp, Artificial Sequence
  Ppargc1alpha reverse primer
cagtttcgttcgacctgcgtaa

SEQ ID NO: 37 - DNA, 20 bp, Artificial Sequence
  Cox8b forward primer
gttcacagtggttcccaaag

SEQ ID NO: 38 - DNA, 20 bp, Artificial Sequence
  Cox8b reverse primer
aacgactatggctgagatcc